(19) United States
(12) Patent Application Publication
(10) Pub. No.: US 2014/0243411 A1
(43) Pub. Date: Aug. 28, 2014

(54) TOPICAL DTPA PRODRUG FORMULATIONS AND METHODS OF USING THE SAME

(71) Applicant: The University of North Carolina at Chapel Hill, Chapel Hill, NC (US)

(72) Inventors: Michael Jay, Chapel Hill, NC (US); Russell Mumper, Chapel Hill, NC (US); Yong Zhang, Durham, NC (US)

(21) Appl. No.: 14/352,344

(22) PCT Filed: Oct. 19, 2012
(86) PCT No.: PCT/US2012/060985

§ 371 Date: Apr. 17, 2014

Related U.S. Application Data
(60) Provisional application No. 61/549,801, filed on Oct. 21, 2011.

(51) Int. Cl. C07C 229/08

(52) U.S. Cl. CPC C07C 229/08
USPC 514/547; 560/169

(57) ABSTRACT

The present invention relates to topical trisodium diethylenetriamine pentaacetic acid (DTPA) prodrug formulations and methods of using the same.

### RELATED APPLICATION DATA

This application claims the benefit of U.S. Provisional Patent Application Ser. No. 61/549,801, filed Oct. 21, 2011, the disclosure of which is incorporated herein by reference in its entirety.

### FIELD OF THE INVENTION

The present invention relates to topical trisodium diethylenetriamine pentaacetic acid (DTPA) prodrug formulations and methods of using topical DTPA formulations.

### BACKGROUND OF THE INVENTION

The United States and many other countries face increasing threats from terrorist groups with respect to the use of weapons of mass destruction against civilian populations. Of particular concern is that some of these groups are intensifying their efforts to acquire and develop nuclear and radiological weapons, and there are a limited number of therapies that can be offered to victims of nuclear terrorism. Currently, the only agents that have been approved by the U.S. Food and Drug Administration (FDA) as chelating agents for americium, curium and plutonium are the calcium and zinc salts of trisodium diethylenetriamine pentaacetic acid (DTPA). Transuranic radionuclides (i.e., those with an atomic number greater than 92), such as americium, curium and plutonium, can potentially be incorporated in radiation dispersal devices (RDDs; “dirty bombs”). The primary goal in treating those exposed to transuranic radionuclides is to chelate the transuranic radionuclides before they become fixed in tissues, such as the liver and bone, and enhance their elimination from contaminated individuals.

The present invention addresses previous shortcomings in the art by providing topical DTPA prodrug formulations and methods of using the same.

### SUMMARY OF THE INVENTION

A first aspect of the invention is a non-aqueous topical formulation comprising:

a compound of Formula (I):

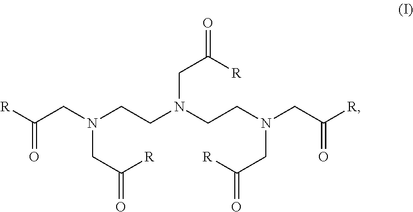

wherein:

R is —OR¹ or —NHR¹;

R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen.

A further aspect of the invention is a topical formulation comprising:

a hydrophobic polymer;

a fatty acid ester; and

a compound of Formula (I):

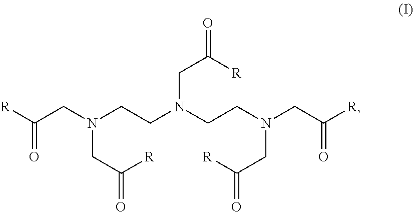

wherein:

R is —OR¹ or —NHR¹;

R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen.

Another aspect of the invention is a method of treating a subject to remove a radioactive element from the subject comprising:

administering a therapeutically effective amount of a topical formulation to a subject, wherein the topical formulation comprises

a compound of Formula (I):

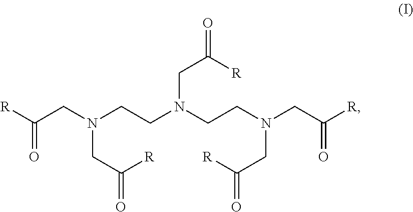

wherein:

R is —OR¹ or —NHR¹;

R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen, and

wherein the topical formulation is non-aqueous.

### BRIEF DESCRIPTION OF THE FIGURES

FIG. 1 shows the in vitro percutaneous permeation profile of a C2E5 nonaqueous gel formulation spiked with [¹⁴C]-C2E5 through Sprague-Dawley (SD) rat skin. The cumulative amounts of C2E5 and its metabolites were calculated based on Equation 4 from [¹⁴C] radioactivity detected in the receiver compartment. Each point represents the mean±SD (n=3).

FIG. 2 shows the cumulative amount of DTPA in the receiver compartment from a C2E5 nonaqueous gel formulation spiked with [¹⁴C]-C2E5 through SD rat skin. The cumulative amounts of DTPA were calculated based on Equation 5 from [¹⁴C] radioactivity detected by HPLC-FSA. Each point represents the mean±SD (n=3).

FIG. 3 shows (A) the total elimination of ²⁴¹Am, (B) daily excretion of ²⁴¹Am in urine, and (C) daily excretion of ²⁴¹Am in feces after a single dose treatment of either: Control (no treatment) (♦), i.v. Ca-DTPA at 14 mg/kg (▪), or transdermal C2E5 gel at 1000 mg/kg(▴).

### DETAILED DESCRIPTION

The present invention will now be described more fully hereinafter. This invention may be embodied in different forms and should not be construed as limited to the embodiments set forth herein. Rather, these embodiments are provided so that this disclosure will be thorough and complete, and will fully convey the scope of the invention to those skilled in the art.

The terminology used in the description herein is for the purpose of describing particular embodiments only and is not intended to be limiting of the invention. As used in the description and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description herein is for the purpose of describing particular embodiments only and is not intended to be limiting of the invention.

All patents, patent applications and publications referred to herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the embodiments of the invention described herein may be used in any combination. For example, features described in relation to one embodiment may also be applicable to and combinable with other embodiments and aspects of the invention.

Moreover, the embodiments of the present invention also contemplate that in some embodiments, any feature or combination of features set forth herein may be excluded or omitted. To illustrate, if the specification states that a complex comprises components A, B and C, in some embodiments, any of A, B or C, or a combination thereof, may be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount or concentration (e.g., the amount of the active agent present in the formulation) and the like, is meant to encompass variations of 10%, 5%, 1%, 0.5%, or even 0.1% of the specified amount.

### Chemical Definitions

“Substituted” as used herein to describe chemical structures, groups, or moieties, refers to the structure, group, or moiety comprising one or more substituents. As used herein, in cases in which a first group is “substituted with” a second group, the second group is attached to the first group whereby a moiety of the first group (typically a hydrogen) is replaced by the second group. The substituted group may contain one or more substituents that may be the same or different.

“Substituent” as used herein references a group that replaces another group in a chemical structure. Typical substituents include nonhydrogen atoms (e.g., halogens), functional groups (such as, but not limited to, amino, sulfhydryl, carbonyl, hydroxyl, alkoxy, carboxyl, silyl, silyloxy, phosphate and the like), hydrocarbyl groups, and hydrocarbyl groups substituted with one or more heteroatoms. Exemplary substituents include, but are not limited to, alkyl, lower alkyl, halo, haloalkyl, alkenyl, alkynyl, cycloalkyl, cycloalkylalkyl, heterocycle, heterocycloalkyl, aryl, arylalkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silylalkyl, silyloxy, boronyl, and modified lower alkyl.

“Alkyl” as used herein alone or as part of another group, refers to a straight or branched chain hydrocarbon containing from 1 to 30 carbon atoms. In some embodiments, the alkyl group may contain 1, 2, or 3 up to 4, 5, 6, 7, 8, 9, 10, 11, 12, 13, 14, 15, 16, 17, 18, 19, 20, 21, 22, 23, 24, 25, 26, 27, 28, 29, or 30 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “alkyl” or “loweralkyl” is intended to include both substituted and unsubstituted alkyl or loweralkyl unless otherwise indicated and these groups may be substituted with groups such as, but not limited to, polyalkylene oxides (such as PEG), halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano, where m=Q, 1, 2 or 3.

“Alkenyl” as used herein alone or as part of another group, refers to a straight or branched chain hydrocarbon containing from 1 to 30 carbon atoms (or in loweralkenyl 1 to 4 carbon atoms) which include 1 to 10 double bonds in the hydrocarbon chain. In some embodiments, the alkenyl group may contain 1, 2, or 3 up to 4, 5, 6, 7, 8, 9, 10, 11, 12, 13, 14, 15, 16, 17, 18, 19, 20, 21, 22, 23, 24, 25, 26, 27, 28, 29, or 30 carbon atoms. Representative examples of alkenyl include, but are not limited to, methylene (═CH₂), vinyl (—CH═CH₂), allyl (—CH₂CH═CH₂), 2-butenyl, 3-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups such as those described in connection with alkyl and loweralkyl above.

“Alkynyl” as used herein alone or as part of another group, refers to a straight or branched chain hydrocarbon containing from 1 to 30 carbon atoms (or in loweralkynyl 1 to 4 carbon atoms) which include at least one triple bond in the hydrocarbon chain. In some embodiments, the alkynyl group may contain 2, or 3 up to 4, 5, 6, 7, 8, 9, 10, 11, 12, 13, 14, 15, 16, 17, 18, 19, 20, 21, 22, 23, 24, 25, 26, 27, 28, 29, or 30 carbon atoms. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralkynyl unless otherwise indicated and these groups may be substituted with the same groups as set forth in connection with alkyl and loweralkyl above.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise.

“Aryl” as used herein alone or as part of another group, refers to a monocyclic carbocyclic ring system or a bicyclic carbocyclic fused ring system or higher having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Amino acid derivative” as used herein, refers to an amino acid substituted with one or more substituents. Exemplary substituents include, but are not limited to, alkyl, lower alkyl, halo, haloalkyl, alkenyl, alkynyl, cycloalkyl, cycloalkylalkyl, heterocyclo, heterocycloalkyl, aryl, arylalkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silylalkyl, silyloxy, boronyl, and modified lower alkyl. Exemplary amino acid derivatives include, but are not limited to, alanine methyl ester, alanine ethyl ester, alanine tert-butyl ester, valine methyl ester, valine ethyl ester, valine tert-butyl ester, phenylalanine methyl ester phenylalanine ethyl ester, phenylalanine tert-butyl ester, phenylalainamide, N-acetyl-phenylalanine, N-ethoxycarbonyl-phenylalanine, tyrosine methyl ester, tyrosine ethyl ester, tyrosine tert-butyl ester, N-acetyl-tyrosine, and O-benzyl-tyrosine.

“Radioactive element” as used herein, refers to a chemical element that emits particulate radiation such as, but not limited to, alpha particles, beta particles, Auger electrons, etc., or a chemical element that emits photons such as, but not limited to, x-rays, gamma rays, etc. The radioactive element may be present in its elemental form or as part of a chemical compound. The radioactive element can have an atomic number of 1 to 103. In certain embodiments of the present invention, the radioactive element is in the actinide series (i.e., has an atomic number of 89-103) of elements. In particular embodiments, the radioactive element is an isotope of plutonium (Pu), americium (Am), or curium (Cm).

### Topical Formulations

The present invention provides topical formulations comprising, consisting essentially of, or consisting of a DTPA (trisodium diethylenetriamine pentaacetic acid) prodrug of Formula (I)

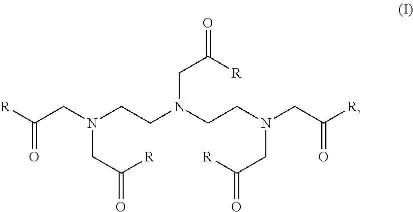

wherein:

R is —OR¹ or —NHR¹;

R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen.

In particular embodiments of the present invention, R is —OR¹ and R¹ is C₁-C₃₀ alkyl. In certain embodiments of the present invention, the DTPA prodrug of Formula (I) is penta-substituted. When one or more R¹ is present, then R¹ at each occurrence may be the same as another R¹ and/or different than another R¹. Thus, all R¹ may be the same, all R¹ may be different, or some R¹ may be the same and some R¹ may be different. A DTPA prodrug of Formula (I) does not include trisodium diethylenetriamine pentaacetic acid (DTPA). Exemplary DTPA prodrugs of Formula (I) and their synthesis can be found in U.S. Pat. No. 8,030,358, which is incorporated herein in its entirety. In some embodiments of the present invention, the DTPA prodrug has the following structure:

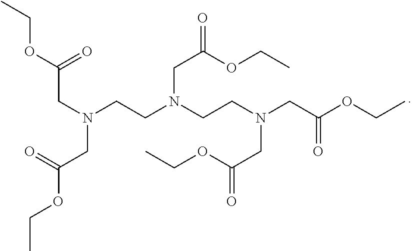 [SMILES: CCOC(=O)CN(CCN(CC(=O)OCC)CC(=O)OCC)CCN(CC(=O)OCC)CC(=O)OCC]

A DTPA prodrug of Formula (I) may be present in a topical formulation of the present invention at a concentration in a range of at least about 5% to about 90% by weight of the formulation or any range and/or individual value therein, such as from about 15% to about 40% or about 30% to about 70%. In certain embodiments of the present invention, a DTPA prodrug of Formula (I) is present in the topical formulation in a concentration of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% by weight of the formulation, or any range therein. In particular embodiments of the present invention, a DTPA prodrug of Formula (I) is present in the topical formulation in a concentration from about 20% to about 55% by weight of the formulation. One or more DTPA prodrugs of Formula (I) may be present in a topical formulation of the present invention, such as 2, 3, 4, or more DTPA prodrugs of Formula (I).

In particular embodiments of the present invention, a DTPA prodrug of Formula (I) has certain physical-chemical properties. For example, the DTPA prodrug of Formula (I) may have a molecular weight from about 400 to about 700 or any range and/or individual value therein, such as from about 400 to about 600 or from about 400 to about 500. In some embodiments of the present invention, the DTPA prodrug of Formula (I) may have a water solubility of about 1.5 mg/mL to about 5.0 mg/mL at a pH in a range of about 6.5 to about 7.5 and of about 15.0 mg/mL to about 25.0 mg/mL at a pH in a range of about 5.0 to about 6.0. Thus, the DTPA prodrug of Formula (I) may have a log P value of about 1 to about 3 at a pH of about 6.0.

According to some embodiments of the present invention, the topical formulation is non-aqueous. The term “non-aqueous” as used herein, refers to a water content from about 0% to about 5% by weight of the formulation or any range and/or individual value therein, such as from about 0.25% to about 1%, about 1% to about 3%, or about 2% to about 5% by weight of the formulation. For example, the water content of a topical formulation of the present invention may be about 5%, 4%, 3%, 2%, 1%, 0.5%, 0.25%, or 0% by weight of the formulation. In certain embodiments of the present invention, the water content of the topical formulation is from about 0 to about 100 ppm or any range and/or individual value therein, such as from about 10 to about 75 ppm or about 30 to about 50 ppm.

Water may be minimally added to a topical formulation of the present invention (i.e., direct addition of water to the topical formulation). Those skilled in the art will appreciate that water may also be physically and/or chemically absorbed at any time during the preparation, storage, and/or use of a topical formulation of the present invention (i.e., indirect addition of water to the topical formulation). In particular embodiments of the present invention, no water is added as a component of the topical formulation before, during, and/or after preparation, storage, and/or use of the topical formulation (i.e., no direct addition of water to the topical formulation). In certain embodiments of the present invention, water is hygroscopically absorbed by the topical formulation before, during, and/or after preparation, storage, and/or use (i.e., indirect addition of water to the topical formulation).

A topical formulation of the present invention may comprise a hydrophobic polymer. The hydrophobic polymer may be a homopolymer and/or a copolymer. One or more hydrophobic polymer(s) may be present in a topical formulation of the present invention, such as 2, 3, 4, or more hydrophobic polymers. Exemplary hydrophobic polymers include, but are not limited to, acrylate polymers, cellulosic polymers, polyamide polymers, polyethylene polymers, poly(vinylpyrrolidone/alkylene) polymers, aminoacrylate polymers, poly(hydroxyester) polymers, poly(alkylene oxide) polymers, poly(ethylene glycol) polymers, and any combination thereof. In particular embodiments of the present invention, the hydrophobic polymer is a cellulosic polymer, such as, but not limited to, a methylcellulose polymer, an ethylcellulose polymer, a carboxymethylcellulose polymer, a hydroxyethyl cellulose polymer, a hydroxyethyl methylcellulose polymer, a hydroxypropyl methylcellulose polymer, a nitrocellulose polymer, and any combination thereof. In certain embodiments of the present invention, an ethylcellulose polymer, such as an ETHOCEL™ ethylcellulose polymer commercially available from The Dow Chemical Company of Midland, Mich., is present in a topical formulation of the present invention.

In some embodiments of the present invention, a topical formulation of the present invention may comprise a hydrophobic polymer with a viscosity range from about 1 to about 500 mPa·s (cP), or any range and/or individual value therein, such as, but not limited to, from about 3 to about 400 cP, about 5 to about 250 cP, about 8 to about 120 cP, about 3 to about 5.5 cP, about 6 to about 8 cP, about 9 to about 11 cP, about 12 to about 16 cP, about 18 to about 22 cP, about 41 to about 49 cP, about 45 to about 55 cP, about 63 to about 77 cP, about 90 to about 110 cP, about 180 to about 220 cP, about 270 to about 330 cP, or about 250 to about 385 cP. The viscosity of a hydrophobic polymer may be measured with a 5% solution of the hydrophobic polymer in a solvent, such as, but not limited to, a solvent comprising 60% toluene and 40% ethanol or 80% toluene and 20% ethanol, at 25° C. in an viscometer, such as, but not limited to a Ubbelohde viscometer. In certain embodiments of the present invention, a hydrophobic polymer with a viscosity from about 8 to about 120 cP is present in a topical formulation of the present invention. In some embodiments of the present invention, an ethylcellulose polymer, such as an ETHOCEL™ ethylcellulose polymer commercially available from The Dow Chemical Company of Midland, Mich., with a viscosity from about 9 to about 11 cP is present in a topical formulation of the present invention.

A topical formulation of the present invention may comprise a hydrophobic polymer at a concentration in a range from about 5% to about 50% by weight of the formulation or any range and/or individual value therein, such as from about 5% to about 35% or about 10% to about 20%. In certain embodiments of the present invention, a hydrophobic polymer is present in the topical formulation in a concentration of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% by weight of the formulation, or any range therein. In particular embodiments of the present invention, the topical formulation comprises a hydrophobic polymer at a concentration from about 10% to about 30% by weight of the formulation.

A topical formulation of the present invention may comprise a fatty acid ester. One or more fatty acid ester(s) may be present in a topical formulation of the present invention, such as 2, 3, 4, or more fatty acid esters. Exemplary fatty acid esters include, but are not limited to, C₆-C₂₂ alkyl and/or alkenyl fatty acid esters such as methyl laurate, ethyl laurate, ethyl myristate, ethyl palmitate, ethyl linoleate, propyl isobutylate, isopropyl laurate, isopropyl myristate, isopropyl palmitate, oleyl myristate, oleyl stearate, and oleyl oleate; ether-esters such as fatty acid esters of ethoxylated fatty alcohols; polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters including sorbitan trioleate and sorbitan monolaurate, and polyoxyethylene sorbitan fatty acid esters including polyoxyethylene (20) sorbitan monolaurate; sucrose fatty acid esters such as saccharose monopalmitate and saccharose monostearate; wax esters such as beeswax, spermaceti, myristyl myristate, stearyl stearate and arachidyl behenate; sterol esters such as cholesterol fatty acid esters, and any combination thereof.

The fatty acid ester may be a glyceride (i.e., a mono-, di-, and/or triglyceride). In particular embodiments of the present invention, the topical formulation comprises a caprylic glyceride and/or a capric glyceride. In some embodiments of the present invention, the topical formulation comprises propylene glycol dicaprylate/dicaprate. In certain embodiments of the present invention, the topical formulation comprises a fatty acid ester such as those commercially available from Sasol of Hamburg, Germany, under the trademark MIGLYOL® and/or IMWITOR®.

A topical formulation of the present invention may comprise a fatty acid ester at a concentration in a range from about 5% to about 90% by weight of the formulation or any range and/or individual value therein, such as from about 10% to about 40% or about 50% to about 80%. In certain embodiments of the present invention, a fatty acid ester is present in the topical formulation in a concentration of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% by weight of the formulation, or any range therein. In particular embodiments of the present invention, the topical formulation comprises a fatty acid ester at a concentration from about 50% to about 75% by weight of the formulation.

A topical formulation of the present invention may optionally include excipients for use in topical formulations known to those of skill in the art and examples may be found in the Handbook of Pharmaceutical Excipients (Rowe, R. C. et al., APhA Publications; 5th ed., 2005). Classes of excipients include, but are not limited to, waxes, emollients, thickening agents/viscosity increasing agents, humectants, pH modifiers, water repelling agents, anti-foaming agents, surfactants, solubilizers, wetting agents, penetration enhancers, colorants, antioxidants, fragrances, and solvents. The excipients may be present in the topical composition at any suitable concentration.

In particular embodiments of the present invention, the topical formulation comprises, consists essentially of, or consists of a DTPA prodrug of Formula (I), a hydrophobic polymer, and/or a fatty acid ester. In certain embodiments of the present invention, the topical formulation is non-aqueous. In some embodiments of the present invention, the topical formulation is homogeneous.

According to some embodiments of the present invention, a topical formulation of the present invention comprises a DTPA prodrug of Formula (I) in an amount of about 20% to about 60% by weight of the formulation, a hydrophobic polymer in an amount of about 10% to about 30% by weight of the formulation, and a fatty acid ester in an amount of about 20% to about 60% by weight of the formulation. In some embodiments of the present invention, a topical formulation of the present invention comprises a DTPA prodrug of Formula (I) having the following structure:

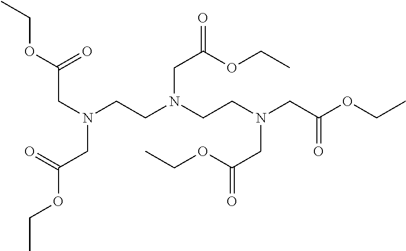 [SMILES: CCOC(=O)CN(CCN(CC(=O)OCC)CC(=O)OCC)CCN(CC(=O)OCC)CC(=O)OCC]

in an amount of about 40% by weight of the formulation, an ethylcellulose polymer in an amount of about 20% by weight of the formulation, and propylene glycol dicaprylate/dicaprate in an amount of about 40% by weight of the formulation.

A topical formulation of the present invention may be in any form suitable for topical administration to body surfaces, including skin, mucous membranes, body cavities, scalp, and nails. A topical formulation of the present invention may be in the form of a solution, an oil, an emulsion, a microemulsion, a suspension, an ointment, a lotion, a gel, a cream, a salve, a paste, a balm, a foam, a suppository, or the like in which the DTPA prodrug of Formula (I) is suspended and/or dissolved. In some embodiments of the present invention, the topical formulation of the present invention is in the form of a spray. In particular embodiments of the present invention, the topical formulation of the present invention is a transdermal formulation. In some embodiments of the present invention, the topical formulation of the present invention is in the form of a transdermal patch, which may comprise a transdermal carrier, such as a polymeric pressure-sensitive adhesive and/or a bioadhesive composition. In other embodiments of the present invention, the topical formulation is in the form of a transdermal implant (e.g., an intradermal implant or a subcutaneous implant). In some embodiments of the present invention, the topical formulation is a homogenous gel that is optionally non-aqueous.

A topical formulation of the present invention may have a density from about 0.25 g/cm³ to about 2 g/cm³, or any range and/or individual value therein, such as, but not limited to, from about 0.5 g/cm³ to about 1.5 g/cm³ or about 1 g/cm³ to about 1.25 g/cm³. In some embodiments of the present invention, a topical formulation of the present invention has a density of about 1 g/cm³.

In some embodiments of the present invention, a topical formulation of the present invention is stable for about 1 week or more, such as, but not limited to, about 2, 3, 4, 5, 6, 7, 8, 9, 10, 11, 12, 13, 14, 15, 16 or more weeks. In certain embodiments of the present invention, a topical formulation of the present invention is stable for about 1 month or more, such as, but not limited to, about 2, 3, 4, 5, 6, 7, 8, 9, 10, 11, 12, 13, 14, 15, 16, 17, 18, or more months. Stability of a topical formulation of the present invention may be measured by storing the topical formulation under accelerated conditions, such as, but not limited to, 40° C., 75% relative humidity (RH), and accessing the stability of the DTPA prodrug of Formula (I) using methods known to those of skill in the field, such as, but not limited to high performance liquid chromatography (HPLC). “Stable”, as used herein, refers to a topical formulation having less than about 20% degradation products of a DTPA prodrug of Formula (I), such as, but not limited to, less than about 15%, 10%, 5%, or 1% degradation products of a DTPA prodrug of Formula (I) and/or the concentration of a DTPA prodrug of Formula (I) in the topical formulation is within about 20% of the initial concentration of the DTPA prodrug of Formula (I) in the topical formulation. In some embodiments of the present invention, a topical formulation of the present invention is stable for at least two months. In certain embodiments of the present invention, at 2 months, no degradation products of a DTPA prodrug of Formula (I) are present in a topical formulation of the present invention and/or there is no statistical difference in the concentration of the DTPA prodrug of Formula (I) in a topical formulation of the present invention compared to the initial concentration of the DTPA prodrug of Formula (I) in the topical formulation.

A topical formulation of the present invention may be made by any suitable method. Exemplary methods of preparing a topical formulation of the present invention include, but are not limited to, a direct mixing method and a solvent evaporation method. The direct mixing method comprises adding each of the components of a topical formulation of the present invention together while mixing and optionally heating one or more components prior to and/or during the addition step. The solvent evaporation method comprises using a solvent to dissolve one or more components of a topical formulation of the present invention and then evaporating the solvent. Exemplary solvents that may be used in the solvent evaporation method include, but are not limited, alcohols, such as ethanol, isobutanol, isopropyl alcohol, benzyl alcohol, propylene glycol, glycerin, sorbitol, xylitol, and any combination thereof.

In some embodiments of the present invention, a topical formulation of the present invention is prepared using the solvent evaporation method. In certain embodiments of the present invention, the solvent evaporation method comprises providing a hydrophobic polymer, optionally drying the hydrophobic polymer, providing a solvent, combining the hydrophobic polymer and a solvent to form a stock solution, providing a fatty acid ester and a DTPA prodrug of Formula (I), combining the fatty acid ester and the DTPA prodrug of Formula (I) with the stock solution, and removing the solvent, thereby producing a topical formulation of the present invention. In certain embodiments of the present invention, a topical formulation of present invention is prepared using the solvent evaporation method using an anhydrous solvent. In some embodiments of the present invention, a method of preparing a topical formulation, such as, but not limited to, the direct mixing method or the solvent evaporation method, provide a homogeneous formulation.

### Methods of Treatment

A further aspect of the present invention provides a method of removing a radioactive element from a subject comprising administering a topical formulation of the present invention. The term “administering”, “administration”, and grammatical variants thereof, as used herein, refer to any mode of topical application to a body surface (e.g., skin, mucous membranes, body cavities, scalp, and/or nails) which results in the physical contact of the formulation with an anatomical site or surface area. Such administration may include applying, spraying, dipping, pressing, squeezing, rolling, rubbing, or the like. Applying may include direct application by finger or swab, or other device such as, but not limited to, a patch. Spraying may include the use of a propellant or mechanical means such as, but not limited to, pump spraying. Other techniques of administration are known to those of skill in the art.

Another aspect of the present invention provides methods of treating a subject comprising administering a topical formulation of the present invention. The term “treating” and grammatical variants thereof, as used herein, refer to any type of topical treatment that imparts a benefit to a subject, including preventing, delaying, and/or reducing the onset and/or progression of one or more symptom(s) and/or condition(s), reducing the severity of one or more symptom(s) and/or condition(s), etc. Those skilled in the art will appreciate that the benefit imparted by the treatment according to the methods of the present invention is not necessarily meant to imply cure or complete prevention (e.g., no detectable incorporation of a radioactive element into a subject's tissue, organs, and/or bones) and/or abolition of the symptom(s) and/or condition(s).

In some embodiments of the present invention, a method of treating a subject exposed to a radioactive element is provided comprising administering a topical formulation of the present invention. Thus, in some embodiments of the present invention, methods are provided for the removal of a radioactive element from a subject exposed to the radioactive element. Those skilled in the art will appreciate that the removal of the radioactive element from the subject may be partial or complete.

In other embodiments of the present invention, a method of treating a subject prior to exposure to a radioactive element is provided comprising administering a topical formulation of the present invention. In a further aspect of the present invention, a method of preventing the incorporation of a radioactive element in a subject is provided comprising administering a topical formulation of the present invention.

“Expose”, “exposure”, and grammatical variants thereof, as used herein, refer to a subject who may come into contact (e.g., a known and/or perceived threat of exposure) and/or coming into contact and/or becoming contaminated (e.g., the subject has internalized and/or incorporated a radioactive element) with ionizing radiation (e.g., alpha particles and/or beta particles) from a radioactive element such that the subject's body may absorb about 100 mrem or more of radiation in one year or less. Thus, the subject may receive an absorbed radiation dose of about 100 mrem, 500 mrem, 1 rem, 5 rem, 10 rem, 30 rem, 50 rem, 100 rem, 250 rem, 500 rem, 1,000 rem or more in one year or less. In some embodiments of the present invention, a subject is contaminated with a radioactive element (e.g., the subject has internalized and/or incorporated a radioactive element). The exposure to the ionizing radiation may be chronic (e.g., occurring over a long duration of time such as month(s) and/or one year) and/or acute (e.g., occurring in a short duration of time such as minute(s), hour(s), and/or day(s)).

The present invention finds use in both veterinary and medical applications. Suitable subjects of the present invention include, but are not limited to avians and mammals. The term “avian” as used herein includes, but is not limited to, chickens, ducks, geese, quail, turkeys, pheasants, ratites (e.g., ostrich), parrots, parakeets, macaws, cockatiels, canaries, finches, and birds in ovo. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), and mammals in utero. In some embodiments of the present invention the subject is a mammal and in certain embodiments the subject is a human. Human subjects include both males and females of all ages including fetal, neonatal, infant, juvenile, adolescent, adult, and geriatric subjects as well as pregnant subjects.

In particular embodiments of the present invention, the subject is “in need of” the methods of the present invention, e.g., the subject has been exposed to a radioactive element, it is believed that the subject will be exposed to a radioactive element, and/or it is believed that the subject has been exposed to a radioactive element. A topical formulation and/or method of the present invention may be particularly suitable for children at or younger than about 10 years of age, such as children at or younger than about 5 years of age or 1 year of age. The present invention may also be particularly suitable for geriatrics.

The administration step may be carried out prior to, during, and/or after exposure to a radioactive element or a threat thereof. The administration step may be carried out to deliver one or more doses of the topical formulation, such as 1, 2, 3, 4, 5, 6, 7, 8, or more doses of the topical formulation. Exemplary dosage regimens include, but are not limited to, once a day, twice a day, every hour, every other day, and any combination thereof for one or more day(s), week(s), month(s), and/or year(s). In certain embodiments of the present invention, the administering step is carried out to remove a radioactive element from a subject. In particular embodiments of the present invention, the administering step is carried out to deliver a therapeutically effective amount of a DTPA prodrug of Formula (I).

As used herein, the term “therapeutically effective amount” refers to an amount of a DTPA prodrug of Formula (I) that elicits a therapeutically useful response in a subject. Those skilled in the art will appreciate that the therapeutic effects need not be complete or curative, as long as some benefit is provided to the subject. In particular embodiments of the present invention, a therapeutically effective amount of a DTPA prodrug results in the detectable elimination or removal of a radioactive element from a subject. Detection of the elimination or removal of a radioactive element may be accomplished by measuring the amount of the radioactive element in the urine, feces, other bodily fluids, and exhaled gas from the lungs of the subject. Methods and instruments used to quantify the amount of a radioactive element removed from a subject are known to those skilled in the art and include, but are not limited to, quantifying the amount of a radioactive element removed using radiation detection equipment such as a gamma scintillation counter, a liquid scintillation counter, a flow scintillation analyzer, an alpha spectrometer, a gas proportional counter, an ionization chamber, a Geiger-Muller counter, etc.

It is appreciated by those in the field that radioactive elements are very poisonous and radiotoxic in the body. “Remove”, “removing”, “removal”, and grammatical variants thereof, as used herein, refer to removing a portion or all of a radioactive element from a subject who may become and/or is contaminated with the radioactive element and may include removing a detectable or nondetectable amount of the radioactive element from the subject. Removing a portion or all of a radioactive element (including removing a detectable or nondetectable amount of the radioactive element) from a subject will generally improve the medical condition of the subject. For example, in some embodiments of the present invention at least about 5%, 10%, 15%, 20%, 25%, 30%, 35%, 40%, 45%, 50%, 55%, 60%, 65%, 70%, 75%, 80%, 85%, 90%, 95%, or more of a radioactive element present in the subject is removed according to the methods of the present invention. Methods for quantifying a subject's exposure level to a radioactive element are known in the art and include, but are not limited to, quantifying the amount of a radioactive element in the area to which a subject was exposed, quantifying by estimating how much of a radioactive element was absorbed or inhaled by a subject, quantifying using whole-body counting instruments, quantifying using external measurements of x-rays emitted from a subject's body, quantifying using physiological based pharmacokinetic models, and quantifying using radioassays of urine, feces, or tissue samples.

According to some embodiments of the present invention, administration of a topical formulation of the present invention to a subject may provide an increase in the amount of a radioactive element removed from the subject as a whole and/or from a particular tissue and/or organ (e.g., liver, kidney, bone, muscle, etc.) of the subject compared to the corresponding amount of the radioactive element removed from the subject if the subject was not administered a treatment to remove the radioactive element and/or to the corresponding amount of the radioactive element removed from the subject if the subject were administered a different treatment and/or different mode of administration to remove the radioactive element (e.g., parenteral administration of DTPA). “Increase”, as used herein in regard to the amount of removal, refers to an improvement in the amount of a radioactive element removed by about 1% or more, such as, but not limited to, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% or more.

In some embodiments of the present invention, administration of a topical formulation of the present invention to a subject may provide sustained levels of DTPA and/or a DTPA metabolite (e.g., a partially substituted DTPA analog) in the subject. In certain embodiments of the present invention, the level of DTPA, a DTPA metabolite, and/or a DTPA prodrug of Formula (I) may vary less than about 30%, 20%, 15%, 10%, or 5% over a period of time, such as, but not limited to, the duration of the topical formulation regimen. In some embodiments of the present invention, sustained levels of DTPA, a DTPA metabolite, and/or a DTPA prodrug of Formula (I) is achieved once the maximum plasma concentration (Cₘₐₓ) of DTPA, a DTPA metabolite, and/or the DTPA prodrug of Formula (I) is achieved. Thus, a topical formulation of the present invention may provide a substantially continuous systemic delivery (i.e., a sustained release) of DTPA, a DTPA metabolite, and/or a DTPA prodrug of Formula (I). In some embodiments of the present invention, the maximum plasma concentration (Cₘₐₓ) of DTPA, a DTPA metabolite, and/or the DTPA prodrug of Formula (I) is achieved at about 1, 2, 3, 4, 5, 6, 7, 8, 9, 10, 11, or 12 hours, or any range therein.

In certain embodiments of the present invention, sustained release of a topical formulation of the present invention may be determined by the length of time an increase in removal of a radioactive element is observed after administration of a topical formulation of the present invention. In some embodiments of the present invention, a single administration of a topical formulation of the present invention to a subject may provide a sustained release of DTPA, a DTPA metabolite, and/or a DTPA prodrug of Formula (I) for at least about 1 day, such as, but not limited to about 2, 3, 4, 5, 6, 7, 8, 9 or more days, or any range therein. In certain embodiments of the present invention, a single administration of a topical formulation of the present invention to a subject may provide a sustained release of DTPA, a DTPA metabolite, and/or a DTPA prodrug of Formula (I) for about 3 days.

The dosage regimen of a topical formulation of the present invention may be adjusted based on the exposure level and/or the subject. In some embodiments of the present invention, the topical formulation regimen is designed to deliver about 5 to about 400 mg of DTPA, a DTPA metabolite, and/or a DTPA prodrug of Formula (I) per kilogram of a subject's total body weight per day. The duration of the administration may be day(s), week(s), month(s), and/or year(s). In certain embodiments of the present invention, the topical formulation is administered until there is no detectable amount and/or removal of a radioactive element for a certain period of time.

In some embodiments of the present invention, administration of a topical formulation of the present invention to a subject may provide a rate of release of a DTPA prodrug of Formula (I) from about 0.001 to about 100 mg/cm², or any range and/or individual value therein, after about 1, 2, 3, 4, 5, 6, 7, 8, 9, 10, 11, or 12 hours, such as, but not limited to, from about 0.001 to about 50 mg/cm², about 0.01 to about 100 mg/cm², about 0.1 to about 1 mg/cm², about 1 to about 100 mg/cm², about 10 to about 100 mg/cm², about 1 to about 7 mg/cm², about 1.5 to about 5 mg/cm², or about 2 to about 4 mg/cm² after about 1, 2, 3, 4, 5, 6, 7, 8, 9, 10, 11, or 12 hours. In certain embodiments of the present invention, administration of a topical formulation of the present invention to a subject may provide a rate of release of a DTPA prodrug of Formula (I) of about 0.001, 0.005, 0.01, 0.05, 0.1, 0.5, 1, 2, 3, 4, 5, 6, 7, 8, 9, 10, 20, 30, 40, 50 60, 70, 80, 90, 100 mg/cm², or any range therein, after about 1, 2, 3, 4, 5, 6, 7, 8, 9, 10, 11, or 12 hours.

In some embodiments of the present invention, administration of a topical formulation of the present invention to a subject may provide an average steady-state flux per unit area of a DTPA prodrug of Formula (I) from about 0.001 to about 20 μmol/cm²/h, or any range and/or individual value therein, such as, but not limited to, from about 0.001 to about 10 μmol/cm²/h, about 0.01 to about 5 μmol/cm²/h, about 0.1 to about 20 μmol/cm²/h, about 1 to about 10 μmol/cm²/h, about 5 to about 20 μmol/cm²/h, about 0.2 to about 1.5 μmol/cm²/h, or about 0.3 to about 1 μmol/cm²/h. In certain embodiments of the present invention, administration of a topical formulation of the present invention to a subject may provide a rate of release of a DTPA prodrug of Formula (I) of about 0.001, 0.005, 0.01, 0.05, 0.1, 0.25, 0.5, 0.75, 1, 1.5, 2, 5, 10, 15, or 20 μmol/cm²/h, or any range therein. In some embodiments, of the present invention, an average steady-state flux per unit area of a DTPA prodrug of Formula (I) may be achieved at about 1, 2, 3, 4, 5, 6, 7, 8, 9, 10, 11, or 12 hours, or any range therein, after administration of a topical formulation of the present invention.

A topical formulation of the present invention may be used alone or in combination with other therapies and/or therapeutic agents. A topical formulation of the present invention may be administered before, during, and/or after administration of another therapy and/or therapeutic agent. In some embodiments of the present invention, a topical formulation of the present invention may be used as a follow-up therapy, such as after parenteral administration of a chelating agent, such as but not limited to DTPA.

The present invention is explained in greater detail in the following non-limiting Examples.

### EXAMPLES

### Example 1

### Ester and Amide Prodrugs of DTPA

More than a dozen esters and amide derivatives of DTPA were synthesized. Preliminary physical-chemical characterization of these compounds was conducted which lead us to select one of these prodrugs, the penta-substituted ethyl ester of DTPA (designated as C2E5, Scheme 1) for further study. C2E5 is an oil with a viscosity of 163 cps at 25° C. This non-thixotropic behavior indicates that the C2E5 fluid properties will remain unchanged by processing during manufacture. The density of C2E5 was determined to be 1.119 g/mL. Equilibrium solubility of C2E5 in aqueous media of varying pH at room temperature was determined to be 3.0 mg/mL at pH 7.1 and 20.3 mg/mL at pH 5.7. The pKa values of the three nitrogen atoms of C2E5 were determined by potentiometric titration to be 3.0, 6.1, and 10.9. The intrinsic partition coefficient (log P) of C2E5 is difficult to measure due to its instability at high pH. Thus, the log D_{6.0} was determined by measuring the partitioning of C2E5 into octanol and water phases. At pH 6.0, the log D was determined to be 2.2, suggesting a highly permeable molecule.

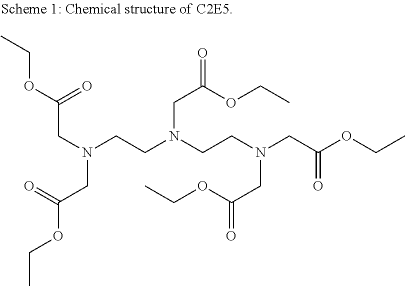 [SMILES: CCOC(=O)CN(CCN(CC(=O)OCC)CC(=O)OCC)CCN(CC(=O)OCC)CC(=O)OCC]

### Example 2

### Development of C2E5 Transdermal Formulations

A number of C2E5 formulations intended for transdermal application were prepared. These included emulsion-based formulations, hydrophilic ointments, and hydrocarbon-based ointments.

The emulsion-based formulations consisted of a water phase (˜85%) comprised of propylene glycol, sorbitol, sorbic acid, butylated hydroxytoluene and simethicone with the pH adjusted to 3.5-4.5, and an oil phase (˜15%) comprised of petrolatum, cetostearyl alcohol, Brij® 58 (Sigma-Aldrich®, St. Louis, Mo.), glyceryl monostearate and polyethylene glycol 400 monostearate. The emulsions were formed by adding the melted oil phase into a vessel containing the water phase and then stirring at 1000 RPM using a mechanical stirrer. The active ingredient, C2E5, was added directly to the emulsion and mixed for 5 minutes at 1000 RPM so that the final concentration of C2E5 in the emulsion ranged from 1% to 16.7% (w/w). Other emulsions were prepared by adding C2E5 to the melted oil phase prior to the formation of the emulsion. Cooling of the emulsions resulted in an off-white cream of uniform consistency.

The hydrophilic ointment formulations consisted of polyethylene glycol (1000-8000), polyethylene glycol 400, stearyl alcohol and butylated hydroxyanisole. The hydrocarbon-based ointments were comprised of white petrolatum, MIGLYOL® 812 (Sasol, Hamburg, Germany), Capmul® MCM (ABITEC, Columbus, Ohio) and butylated hydroxyanisole. For both of these ointments, C2E5 was added directly to the ointment and mixed for 5 minutes at 1000 RPM using a mechanical stirrer so that the final concentration of C2E5 in the ointments ranged from 5% to 20% (w/w).

The stability of C2E5 in the emulsion-based formulations after storage under accelerated conditions (40° C., 75% relative humidity (RH)) was inadequate, likely to due to ester hydrolysis. Thus, a series of non-aqueous gel formulations containing C2E5 were subsequently prepared, and the stability of C2E5 in these formulations was assessed.

### Example 3

### Preparation of C2E5 Nonaqueous Gel Formulations

Gels composed of different grades of ethylcellulose (EC) polymers (ETHOCEL™ Standard 7 FP (EC7), ETHOCEL™ Standard 10 FP (EC 10), or ETHOCEL™ Standard 100 FP (EC 100) available from The Dow Chemical Company of Midland, Mich. and esters of fatty acids (MIGLYOL® 810, 812, 818, 829, 840 or 8810 or IMWITOR® 808 or 8108 available from Sasol of Hamburg, Germany) were prepared by mixing 0.2-1.8 g of EC and 0.0-1.8 g of MIGLYOL® or IMWITOR® and heating to 60° C., The ECs were dissolved in the hot MIGLOYL®/IMWITOR® under continuous vortexing (Tables 1-24).

TABLE 1
Nonaqueous gel formulations containing ETHOCEL ™ Standard 7 FP
(EC7) and optionally MIGLOYL ® 840 and C2E5.

| | EC7 | % w/w | MIGLOYL ® | % w/w | C2E5 | % w/w |
|---|---|---|---|---|---|---|
| Sample name | (g) | (EC7/total) | 840 (g) | (MIGLOYL ®/total) | (g) | (C2E5/total) |
| Formulation 1 | 0.2 | 10% | 0 | 0% | 1.8 | 90% |
| Formulation 2 | 0.2 | 10% | 0.2 | 10% | 1.6 | 80% |
| Formulation 3 | 0.2 | 10% | 0.6 | 30% | 1.2 | 60% |
| Formulation 4 | 0.2 | 10% | 1.0 | 50% | 0.8 | 40% |
| Formulation 5 | 0.2 | 10% | 1.4 | 70% | 0.4 | 20% |
| Formulation 6 | 0.2 | 10% | 1.8 | 90% | 0 | 0% |
| Formulation 7 | 0.4 | 20% | 0 | 0% | 1.6 | 80% |
| Formulation 8 | 0.4 | 20% | 0.4 | 20% | 1.2 | 60% |
| Formulation 9 | 0.4 | 20% | 0.8 | 40% | 0.8 | 40% |
| Formulation 10 | 0.4 | 20% | 1.2 | 60% | 0.4 | 20% |
| Formulation 11 | 0.4 | 20% | 1.6 | 80% | 0 | 0% |
| Formulation 12 | 0.6 | 30% | 0 | 0% | 1.4 | 70% |
| Formulation 13 | 0.6 | 30% | 0.2 | 10% | 1.2 | 60% |
| Formulation 14 | 0.6 | 30% | 0.6 | 30% | 0.8 | 40% |
| Formulation 15 | 0.6 | 30% | 1.0 | 50% | 0.4 | 20% |
| Formulation 16 | 0.6 | 30% | 1.4 | 70% | 0 | 0% |
| Formulation 17 | 1.0 | 20% | 4.0 | 80% | 0 | 0 |
| Formulation 18 | 1.5 | 30% | 3.5 | 70% | 0 | 0 |
| Formulation 19 | 1.0 | 16% | 4.0 | 64% | 1.25 | 20% |
| Formulation 20 | 1.5 | 21% | 3.5 | 49% | 2.14 | 30% |
| Formulation 21 | 0.5 | 8% | 4.5 | 72% | 1.25 | 20% |
| Formulation 22 | 0.5 | 7% | 4.5 | 63% | 2.14 | 30% |
| Formulation 23 | 1.0 | 16% | 4.0 | 64% | 1.25 | 20% |
| Formulation 24 | 1.0 | 14% | 4.0 | 56% | 2.14 | 30% |

TABLE 2
Nonaqueous gel formulations containing ETHOCEL ™ Standard 10 FP
(EC10) and optionally MIGLOYL ® 840 and C2E5.

| | EC10 | % w/w | MIGLOYL ® | % w/w | C2E5 | % w/w |
|---|---|---|---|---|---|---|
| Sample name | (g) | (EC10/total) | 840 (g) | (MIGLOYL ®/total) | (g) | (C2E5/total) |
| Formulation 1 | 0.2 | 10% | 0 | 0% | 1.8 | 90% |
| Formulation 2 | 0.2 | 10% | 0.2 | 10% | 1.6 | 80% |
| Formulation 3 | 0.2 | 10% | 0.6 | 30% | 1.2 | 60% |
| Formulation 4 | 0.2 | 10% | 1.0 | 50% | 0.8 | 40% |
| Formulation 5 | 0.2 | 10% | 1.4 | 70% | 0.4 | 20% |
| Formulation 6 | 0.2 | 10% | 1.8 | 90% | 0 | 0% |
| Formulation 7 | 0.4 | 20% | 0 | 0% | 1.6 | 80% |
| Formulation 8 | 0.4 | 20% | 0.4 | 20% | 1.2 | 60% |
| Formulation 9 | 0.4 | 20% | 0.8 | 40% | 0.8 | 40% |
| Formulation 10 | 0.4 | 20% | 1.2 | 60% | 0.4 | 20% |
| Formulation 11 | 0.4 | 20% | 1.6 | 80% | 0 | 0% |
| Formulation 12 | 0.6 | 30% | 0 | 0% | 1.4 | 70% |
| Formulation 13 | 0.6 | 30% | 0.2 | 10% | 1.2 | 60% |
| Formulation 14 | 0.6 | 30% | 0.6 | 30% | 0.8 | 40% |
| Formulation 15 | 0.6 | 30% | 1.0 | 50% | 0.4 | 20% |
| Formulation 16 | 0.6 | 30% | 1.4 | 70% | 0 | 0% |
| Formulation 17 | 0.5 | 10% | 4.5 | 90% | 0 | 0 |
| Formulation 18 | 0.75 | 15% | 4.25 | 85% | 0 | 0 |
| Formulation 19 | 1.125 | 16% | 4.5 | 64% | 1.43 | 20% |
| Formulation 20 | 1.82 | 24% | 4.25 | 46% | 1.52 | 20% |
| Formulation 21 | 0.5 | 8% | 4.5 | 72% | 1.25 | 20% |
| Formulation 22 | 0.5 | 7% | 4.5 | 63% | 2.14 | 30% |
| Formulation 23 | 0.75 | 12% | 4.25 | 68% | 1.25 | 20% |
| Formulation 24 | 0.75 | 10% | 4.25 | 60% | 2.14 | 30% |

TABLE 3
Nonaqueous gel formulations containing ETHOCEL ™ Standard 10 FP
(EC10), MIGLOYL ® 840, C2E5, and α-tocopherol.

| | EC 10 | % w/w | MIGLOYL ® | % w/w | C2E5 | % w/w | α-tocopherol |
|---|---|---|---|---|---|---|---|
| Sample name | (g) | (EC10/total) | 840 (g) | (MIGLOYL ®/total) | (g) | (C2E5/total) | (mg) |
| Formulation 1 | 0.4 | 20% | 0.8 | 40% | 0.8 | 40% | 20 |
| Formulation 2 | 0.4 | 20% | 0.8 | 40% | 0.8 | 40% | 40 |

TABLE 4
Nonaqueous gel formulations containing ETHOCEL ™ Standard 100 FP
(EC100) and optionally MIGLOYL ® 840 and C2E5.

| | EC100 | % w/w | MIGLOYL ® | % w/w | C2E5 | % w/w |
|---|---|---|---|---|---|---|
| Sample name | (g) | (EC100/total) | 840 (g) | (MIGLOYL ®/total) | (g) | (C2E5/total) |
| Formulation 1 | 0.2 | 10% | 0 | 0% | 1.8 | 90% |
| Formulation 2 | 0.2 | 10% | 0.2 | 10% | 1.6 | 80% |
| Formulation 3 | 0.2 | 10% | 0.6 | 30% | 1.2 | 60% |
| Formulation 4 | 0.2 | 10% | 1.0 | 50% | 0.8 | 40% |
| Formulation 5 | 0.2 | 10% | 1.4 | 70% | 0.4 | 20% |
| Formulation 6 | 0.2 | 10% | 1.8 | 90% | 0 | 0% |
| Formulation 7 | 0.4 | 20% | 0 | 0% | 1.6 | 80% |
| Formulation 8 | 0.4 | 20% | 0.4 | 20% | 1.2 | 60% |
| Formulation 9 | 0.4 | 20% | 0.8 | 40% | 0.8 | 40% |
| Formulation 10 | 0.4 | 20% | 1.2 | 60% | 0.4 | 20% |
| Formulation 11 | 0.4 | 20% | 1.6 | 80% | 0 | 0% |
| Formulation 12 | 0.6 | 30% | 0 | 0% | 1.4 | 70% |
| Formulation 13 | 0.6 | 30% | 0.2 | 10% | 1.2 | 60% |
| Formulation 14 | 0.6 | 30% | 0.6 | 30% | 0.8 | 40% |
| Formulation 15 | 0.6 | 30% | 1.0 | 50% | 0.4 | 20% |
| Formulation 16 | 0.6 | 30% | 1.4 | 70% | 0 | 0% |
| Formulation 17 | 0.5 | 10% | 4.5 | 90% | 0 | 0 |
| Formulation 18 | 0.75 | 15% | 4.25 | 85% | 0 | 0 |
| Formulation 19 | 0.5 | 8% | 4.5 | 72% | 1.26 | 20% |
| Formulation 20 | 0.75 | 12% | 4.25 | 68% | 1.25 | 20% |
| Formulation 21 | 0.5 | 8% | 4.5 | 72% | 1.25 | 20% |
| Formulation 22 | 0.5 | 7% | 4.5 | 63% | 2.14 | 30% |
| Formulation 23 | 0.75 | 12% | 4.25 | 68% | 1.25 | 20% |
| Formulation 24 | 0.75 | 10% | 4.25 | 60% | 2.14 | 30% |

TABLE 5
Nonaqueous gel formulations containing ETHOCEL ™ Standard
7 FP (EC7) and IMWITOR ® 808.

| | | % w/w | | % w/w |
|---|---|---|---|---|
| | EC7 | (EC7/ | IMWITOR ® | (IMWITOR ®/ |
| Sample name | (g) | total) | 808 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 6
Nonaqueous gel formulations containing ETHOCEL ™ Standard
10 FP (EC10) and IMWITOR ® 808.

| | | % w/w | | % w/w |
|---|---|---|---|---|
| | EC10 | (EC10/ | IMWITOR ® | (IMWITOR ®/ |
| Sample name | (g) | total) | 808 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 6
Nonaqueous gel formulations containing ETHOCEL ™ Standard
100 FP (EC100) and IMWITOR ® 808.

| | | % w/w | | % w/w |
|---|---|---|---|---|
| | EC100 | (EC100/ | IMWITOR ® | (IMWITOR ®/ |
| Sample name | (g) | total) | 808 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 7
Nonaqueous gel formulations containing ETHOCEL ™
Standard 7 FP (EC7) and IMWITOR ® 8108.

| | | % w/w | | % w/w |
|---|---|---|---|---|
| | EC7 | (EC7/ | IMWITOR ® | (IMWITOR ®/ |
| Sample name | (g) | total) | 8108 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 8
Nonaqueous gel formulations containing ETHOCEL ™ Standard
10 FP (EC10) and IMWITOR ® 8108.

| | | % w/w | | % w/w |
|---|---|---|---|---|
| | EC10 | (EC10/ | IMWITOR ® | (IMWITOR ®/ |
| Sample name | (g) | total) | 8108 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 9
Nonaqueous gel formulations containing ETHOCEL ™ Standard
100 FP (EC100) and IMWITOR ® 8108.

| | | % w/w | | % w/w |
|---|---|---|---|---|
| | EC100 | (EC100/ | IMWITOR ® | (IMWITOR ®/ |
| Sample name | (g) | total) | 8108 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 10
Nonaqueous gel formulations containing ETHOCEL ™ Standard
7 FP (EC7) and MIGLOYL ® 810.

| | | % w/w | | % w/w |
|---|---|---|---|---|
| | EC7 | (EC7/ | MIGLOYL ® | (MIGLOYL ®/ |
| Sample name | (g) | total) | 810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 11
Nonaqueous gel formulations containing ETHOCEL ™ Standard
10 FP (EC10) and MIGLOYL ® 810.

| | | % w/w | | % w/w |
|---|---|---|---|---|
| | EC10 | (EC10/ | MIGLOYL ® | (MIGLOYL ®/ |
| Sample name | (g) | total) | 810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 12
Nonaqueous gel formulations containing ETHOCEL ™ Standard
100 FP (EC100) and MIGLOYL ® 810.

| | | % w/w | | % w/w |
|---|---|---|---|---|
| | EC100 | (EC100/ | MIGLOYL ® | (MIGLOYL ®/ |
| Sample name | (g) | total) | 810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 13
Nonaqueous gel formulations containing ETHOCEL ™ Standard
7 FP (EC7) and MIGLOYL ® 812.

| | | % w/w | | % w/w |
|---|---|---|---|---|
| | EC7 | (EC7/ | MIGLOYL ® | (MIGLOYL ®/ |
| Sample name | (g) | total) | 812 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 14
Nonaqueous gel formulations containing ETHOCEL ™ Standard
10 FP (EC10) and MIGLOYL ® 812.

| | | % w/w | | % w/w |
|---|---|---|---|---|
| | EC10 | (EC10/ | MIGLOYL ® | (MIGLOYL ®/ |
| Sample name | (g) | total) | 812 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 15
Nonaqueous gel formulations containing ETHOCEL ™ Standard
100 FP (EC100) and MIGLOYL ® 812.

| | | % w/w | | % w/w |
|---|---|---|---|---|
| | EC100 | (EC100/ | MIGLOYL ® | (MIGLOYL ®/ |
| Sample name | (g) | total) | 812 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 16
Nonaqueous gel formulations containing ETHOCEL ™ Standard
7 FP (EC7) and MIGLOYL ® 818.

| | | % w/w | | % w/w |
|---|---|---|---|---|
| | EC7 | (EC7/ | MIGLOYL ® | (MIGLOYL ®/ |
| Sample name | (g) | total) | 818 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 17
Nonaqueous gel formulations containing ETHOCEL ™ Standard
10 FP (EC10) and MIGLOYL ® 818.

| | | % w/w | | % w/w |
|---|---|---|---|---|
| | EC10 | (EC10/ | MIGLOYL ® | (MIGLOYL ®/ |
| Sample name | (g) | total) | 818 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 18
Nonaqueous gel formulations containing ETHOCEL ™ Standard
100 FP (EC100) and MIGLOYL ® 818.

| | | % w/w | | % w/w |
|---|---|---|---|---|
| | EC100 | (EC100/ | MIGLOYL ® | (MIGLOYL ®/ |
| Sample name | (g) | total) | 818 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 19
Nonaqueous gel formulations containing ETHOCEL ™ Standard
7 FP (EC7) and MIGLOYL ® 829.

| | | % w/w | | % w/w |
|---|---|---|---|---|
| | EC7 | (EC7/ | MIGLOYL ® | (MIGLOYL ®/ |
| Sample name | (g) | total) | 829 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 20
Nonaqueous gel formulations containing ETHOCEL ™ Standard
10 FP (EC10) and MIGLOYL ® 829.

| | | % w/w | | % w/w |
|---|---|---|---|---|
| | EC10 | (EC10/ | MIGLOYL ® | (MIGLOYL ®/ |
| Sample name | (g) | total) | 829 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 21
Nonaqueous gel formulations containing ETHOCEL ™ Standard
100 FP (EC100) and MIGLOYL ® 829.

| | | % w/w | | % w/w |
|---|---|---|---|---|
| | EC100 | (EC100/ | MIGLOYL ® | (MIGLOYL ®/ |
| Sample name | (g) | total) | 829 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 22
Nonaqueous gel formulations containing ETHOCEL ™
Standard 7 FP (EC7) and MIGLOYL ® 8810.

| | | % w/w | | % w/w |
|---|---|---|---|---|
| | EC7 | (EC7/ | MIGLOYL ® | (MIGLOYL ®/ |
| Sample name | (g) | total) | 8810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 23
Nonaqueous gel formulations containing ETHOCEL ™ Standard
10 FP (EC10) and MIGLOYL ® 8810.

| | | % w/w | | % w/w |
|---|---|---|---|---|
| | EC10 | (EC10/ | MIGLOYL ® | (MIGLOYL ®/ |
| Sample name | (g) | total) | 8810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

TABLE 24
Nonaqueous gel formulations containing ETHOCEL ™ Standard
100 FP (EC100) and MIGLOYL ® 8810.

| | | % w/w | | % w/w |
|---|---|---|---|---|
| | EC100 | (EC100/ | MIGLOYL ® | (MIGLOYL ®/ |
| Sample name | (g) | total) | 8810 (g) | total) |
| Formulation 1 | 0.2 | 10 | 1.8 | 90 |
| Formulation 2 | 0.4 | 20 | 1.6 | 80 |
| Formulation 3 | 0.6 | 30 | 1.4 | 70 |

ETHOCEL™ ethylcellulose resins are versatile, organosoluble, thermoplastic polymers. They result from the reaction of ethyl chloride with alkali cellulose. Product names are based on different degrees of ethoxyl content of the modified cellulose. MIGLYOL® neutral oils are clear, slightly yellowish esters of saturated coconut and palmkernel oil-derived caprylic and capric fatty acids and glycerin or propylene glycol. IMWITOR® glyceryl stearates are esters of the natural fatty acids, stearic and palmitic acid, with glycerin.

The mixtures that formed homogenous gels were subsequently used for the preparation of C2E5 nonaqueous gel formulations in which C2E5 was added under a nitrogen overlay with continuous vortexing so that the C2E5 concentration varied in the final gel from 20-90% (w/w). These formulations all contained MIGLYOL® 840, a propylene glycol diester of saturated vegetable fatty acids with chain lengths of C8 and C10. MIGLOYL® neutral oils are non-occlusive oils with excellent spreadability and emollient properties. In addition, they have penetration-enhancing lipid bases and are a fat component, readily miscible with natural oils and surfactants.

Nonaqueous gel transdermal C2E5 formulations were prepared by mixing 0.2-0.6 g of EC and 0.0-1.8 g of MIGLYOL® 840, and heating to 60° C. The ECs were dissolved in the hot MIGLYOL® 840 under continuous vortexing to form homogenous gels. To the gels were added 0.0-1.8 g of C2E5 under a nitrogen overlay with continuous vortexing to yield homogenous gels. The formulations that possessed the best properties including physical appearance, uniformity, C2E5 concentration and ease of manufacture were further evaluated for stability of C2E5 after storage at 40° C./75% RH. The selected formulations appear in Table 25:

TABLE 25
Composition of nonaqueous gel transdermal C2E5 formulations.

| | | % | | % | | % |
|---|---|---|---|---|---|---|
| | | w/w | | w/w | | w/w |
| Sample | EC | (EC/ | MIGLOYL ® | (MIGLOYL ®/ | C2E5 | (C2E5/ |
| name | (g) | total) | 840 (g) | total) | (g) | total) |
| Gel 7 - | EC7 | 20% | 0.8 | 40% | 0.8 | 40% |
| 20/40/ | 0.4 | | | | | |
| 40 | | | | | | |
| Gel 7 - | EC7 | 30% | 0.6 | 30% | 0.8 | 40% |
| 30/30/ | 0.6 | | | | | |
| 40 | | | | | | |
| Gel 10 - | EC10 | 20% | 1.2 | 60% | 0.4 | 20% |
| 20/60/ | 0.4 | | | | | |
| 20 | | | | | | |
| Gel 10 - | EC10 | 30% | 1.0 | 50% | 0.4 | 20% |
| 30/50/ | 0.6 | | | | | |
| 20 | | | | | | |

Crimped vials containing the hot gels were subsequently transferred to an incubator (40° C., 75% RH) for storage. Selected samples were withdrawn at 0, 7 and 13 weeks for HPLC analysis. The details of the HPLC method are provided in Table 26:

TABLE 26
HPLC method details.

| | |
|---|---|
| Separation Column | Chromolith ® Fast Gradient Column, RP-18e, |
| | 50-2 mm |
| Guard Column | Alltima C18 5μ |
| Mobile Phase | Acetonitrile/0.1% Trifluoroacetic Acid |
| (Organic) | |
| Mobile Phase | Water/0.1% Trifluoroacetic Acid |
| (Water) | |
| Flow rate | 0.2 mL/min |
| Detector | Evaporative Light Scattering Detector (ELSD) |
| Run Time | 26 min |

A gradient mobile phase system was used by changing from 5% of acetonitrile/0.1% trifluoroacetic acid: 95% of water/0.1% trifluoroacetic acid to 95% of acetonitrile/0.1% trifluoroacetic acid: 5% of water/0.1% trifluoroacetic acid over 10 minutes. The mobile phase was then held at 95% of acetonitrile/0.1% trifluoroacetic acid: 5% of water/0.1% trifluoroacetic acid for another 10 minutes. The gradient was then returned to 5% of acetonitrile/0.1% trifluoroacetic acid: 95% of water/0.1% trifluoroacetic acid for 3 minutes followed by holding the mobile phase of acetonitrile/0.1% trifluoroacetic acid: 95% of water/0.1% trifluoroacetic acid for an additional 3 minutes.

### Example 4

### Stability of the Nonaqueous Gel Formulations

The percent of intact C2E5 in various nonaqueous gel formulations was evaluated (Tables 27-29). The stability of the formulations after storage under accelerated conditions (40° C., 75% RH) for three months is reported in the Table 30.

TABLE 27
C2E5 nonaqueous gel formulations evaluated
containing ETHOCEL ™ Standard 7
FP (EC7) and MIGLOYL ® 840.

| | | MIGLOYL ® | | | |
|---|---|---|---|---|---|
| | EC7 (g) | 840 (g) | % w/w | C2E5 (g) | % w/w |
| Sample | (Actually | (Actually | (EC7/ | (Actually | (C2E5/ |
| name | added wt.) | added wt.) | total) | added wt.) | total) |
| YZ4- | 0.5 (0.501) | 4.5 (4.500) | 7.8 | 1.25 (1.375) | 21.5 |
| 29-1 | | | | | |
| YZ4- | 0.5 (0.503) | 4.5 (4.508) | 7.0 | 2.14 (2.147) | 30.0 |
| 29-2 | | | | | |
| YZ4- | 1.0 (1.003) | 4.0 (4.000) | 16.0 | 1.25 (1.253) | 20.0 |
| 29-3 | | | | | |
| YZ4- | 1.0 (1.001) | 4.0 (4.000) | 14.0 | 2.14 (2.142) | 30.0 |
| 29-4 | | | | | |

TABLE 28
C2E5 nonaqueous gel formulations evaluated
containing ETHOCEL ™ Standard 10
FP (EC10) and MIGLOYL ® 840.

| | | MIGLOYL ® | | | |
|---|---|---|---|---|---|
| | EC10 (g) | 840 (g) | % w/w | C2E5 (g) | % w/w |
| Sample | (Actually | (Actually | (EC10/ | (Actually | (C2E5/ |
| name | added wt.) | added wt.) | total) | added wt.) | total) |
| YZ4- | 0.5 (0.503) | 4.5 (4.507) | 8.0 | 1.25 (1.252) | 20.0 |
| 29-5 | | | | | |
| YZ4- | 0.5 (0.503) | 4.5 (4.505) | 7.0 | 2.14 (2.183) | 30.4 |
| 29-6 | | | | | |
| YZ4- | 0.75 (0.753) | 4.25 (4.250) | 11.6 | 1.25 (1.459) | 22.6 |
| 29-7 | | | | | |
| YZ4- | 0.75 (0.753) | 4.25 (4.254) | 10.5 | 2.14 (2.157) | 30.1 |
| 29-8 | | | | | |

TABLE 29
C2E5 nonaqueous gel formulations evaluated
containing ETHOCEL ™ Standard 100
FP (EC100) and MIGLOYL ® 840.

| | | MIGLOYL ® | | | |
|---|---|---|---|---|---|
| | EC100 (g) | 840 (g) | % w/w | C2E5 (g) | % w/w |
| Sample | (Actually | (Actually | (EC100/ | (Actually | (C2E5/ |
| name | added wt.) | added wt.) | total) | added wt.) | total) |
| YZ4- | 0.5 (0.500) | 4.5 (4.500) | 8.0 | 1.25 (1.262) | 20.1 |
| 29-9 | | | | | |
| YZ4- | 0.5 (0.504) | 4.5 (4.500) | 7.0 | 2.14 (2.144) | 30.2 |
| 29-10 | | | | | |
| YZ4- | 0.75 (0.752) | 4.25 (4.252) | 12.0 | 1.25 (1.254) | 20.2 |
| 29-11 | | | | | |
| YZ4- | 0.75 (0.750) | 4.25 (4.251) | 10.5 | 2.14 (2.165) | 30.2 |
| 29-12 | | | | | |

TABLE 30
Stability of C2E5 nonaqueous gel formulations (% intact C2E5)
after storage for three months under accelerated conditions.

| | Original C2E5 | Remaining C2E5 | % of | Standard devi- |
|---|---|---|---|---|
| | concentration | concentration | C2E5 | ation of % |
| Sample | of samples | of samples | remain- | of C2E5 |
| name | (mg/ml) | (mg/ml) | ing | remaining |
| YZ4- | 0.546 | 0.484 | 88.7 | 7.9 |
| 29-1 | | | | |
| YZ4- | 0.812 | 0.743 | 91.5 | 2.4 |
| 29-2 | | | | |
| YZ4- | 0.571 | 0.490 | 85.8 | 6.2 |
| 29-3 | | | | |
| YZ4- | 0.737 | 0.628 | 85.3 | 7.0 |
| 29-4 | | | | |
| YZ4- | 0.520 | 0.448 | 86.2 | 9.8 |
| 29-5 | | | | |
| YZ4- | 0.755 | 0.680 | 90.0 | 10.5 |
| 29-6 | | | | |
| YZ4- | 0.545 | 0.481 | 88.2 | 10.8 |
| 29-7 | | | | |
| YZ4- | 0.781 | 0.680 | 87.1 | 18.5 |
| 29-8 | | | | |
| YZ4- | 0.534 | 0.471 | 88.2 | 8.8 |
| 29-9 | | | | |
| YZ4- | 0.773 | 0.806 | 104.3 | 5.5 |
| 29-10 | | | | |
| YZ4- | 0.503 | 0.548 | 108.9 | 8.2 |
| 29-11 | | | | |
| YZ4- | 0.854 | 0.696 | 81.6 | 3.8 |
| 29-12 | | | | |

Thus, the C2E5 nonaqueous gel formulations are stable for a sufficient period of time (at least 13 weeks) at accelerated temperatures, which predicts significant shelf-lives when stored at room temperature.

### Example 5

### Efficacy of Transdermal C2E5

Anesthesia (isoflurane, 2% by vaporizer) was induced in 12 female Sprague-Dawley rats (240 g-420 g), and the dorsal skin above the cervical vertebrae and anterior thoracic vertebrae was exposed by shaving.

Contamination: All animals were contaminated with ²⁴¹Am Nitrate (250 nCi, 0.1 mL) via an intramuscular injection while under isoflurane anesthesia.

Nonaqueous gel formulations of C2E5 (Table 31) were applied to the shaved area of rats immediately after contamination with an intramuscular injection of ²⁴¹Am Nitrate. The cut off top of a 3 mL syringe (approx area 1 cm²) was pressed onto the skin around the application area to ensure that the dose was contained at the application site until absorbed. Cotton buds were used as applicators for non-aqueous gel treatments (200 mg/kg C2E5). The gels were applied to the prepared skin; after approximately 3 min to allow the gel to dry, anesthesia was withdrawn. The mass of gel applied was recorded for each animal to permit actual dose determination. Animals were euthanized 7 days after contamination, except untreated animals which were euthanized 12 days after contamination.

TABLE 31
Transdermal efficacy of nonaqueous gel formulations
containing ETHOCEL ™ Standard 7 FP (EC7),
10 FP (EC10), or 100 FP (EC100) and MIGLYOL ® 840 and C2E5.

| Formulation | Ethylcellulose (%) | MIGLYOL ® 840 (%) | C2E5 (%) |
|---|---|---|---|
| Formulation 1 | EC7 7% | 63% | 30% |
| Formulation 2 | EC10 7% | 63% | 30% |
| Formulation 3 | EC100 7% | 63% | 30% |

Urine and feces were collected daily; metabolic cages were washed after euthanasia. The ²⁴¹Am content of all urine, feces and cage washing samples were determined using a gamma counter (2470 or 2480 Wizard2, Perkin Elmer).

The decorporation efficacy of the gels was assessed by adding the total amount of ²⁴¹Am in the urine, feces and cage washings and dividing by the amount of ²⁴¹Am administered to the animals. The results, expressed as the percent of the radioactivity collected in urine, feces and cage washings as a percent of the injected dose of radioactivity are shown in Table 32.

TABLE 32
Percentage of ²⁴¹Am eliminated at seven days post-contamination.

| | % of injected ²⁴¹Am eliminated |
|---|---|
| Treatment | (mean ± SD) |
| Untreated n = 4 | 9.2 ± 2.8 |
| Formulation 1, n = 2 | 17.5 ± 5.6 |
| Formulation 2, n = 2 | 15.9 ± 4.9 |
| Formulation 3, n = 2 | 18.2 ± 0.2 |

Thus, the amount of radioactivity eliminated from contaminated rats following a single topical treatment of a C2E5 nonaqueous gel formulation was nearly twice that of untreated animals.

### Example 6

### Pre aratisonofNon-Aqueous Gels Containing MIGLYOL® 840

### Methods

Direct mixing method: MIGLYOL® 840 and C2E5 were first heated to 60° C. followed by the slow addition of fine particles of ethyl cellulose (EC7, EC 10 or EC 100) into the fatty acid ester under constant stirring. The EC, MIGLYOL® 840, and C2E5 mixtures were held under stirring until the mixtures turned into clear, viscous solutions. Non-aqueous gels were formed after cooling to ambient temperature. Four formulations were prepared from each EC polymer (Table 33). The non-aqueous gel samples were put under vacuum to remove air bubbles trapped in the gels.

Solvent evaporation method: Only EC10 was used to prepare a non-aqueous gel using the solvent evaporation method. The EC10 material was dried at 60° C. for ˜24 h before use in gel preparations. Pre-dried EC10 particles were initially dissolved in anhydrous ethanol (10% w/v of EC10 to ethanol) to form the EC10 stock solution. The C2E5 non-aqueous gel was prepared by adding EC 10 stock solution, MIGLYOL® 840, and C2E5 to form a homogenous solution, followed by removing ethanol under vacuum. When the gel was less than 102% of the theoretical weight, the C2E5 non-aqueous gels were transferred into a storage container under nitrogen and sealed with an airtight cap, covered with aluminum foil to protect from light, and stored at 4° C. for future use.

The C2E5 concentration in these gel formulations was determined using a Shimadzu Prominence high pressure liquid chromatography (HPLC) system equipped with an Alltech 3300 evaporative light scattering detector (ELSD). A reverse phase gradient separation was performed using an Alltima C18 column (250×2.1 mm I.D., 5 μm) at 40° C. and a flow rate of 0.25 mL/min. The solvents that comprised the mobile phase were water with 0.1% TFA (A), MeCN (B), and IPA (C). The linear gradient for the mobile phase mixture (A:B:C) was from 94:4:2 to 25:50:25 over 35 min, followed by a change to 0:0:100 in 0.5 min and an equilibration phase of 0:0:100 for 9.5 min and ending with a reversal to 94:4:2 in 0.5 min and an equilibration phase of 94:4:2 for 9.5 min. The ELSD was operated at 40° C. with 1.9 L/min nitrogen gas flow. Triplicate injections for each sample of C2E5 non-aqueous gel dissolved in anhydrous ethanol were performed with a volume of 10 μL per injection, and the retention time of C2E5 was 26 min. Samples were held at ambient temperature during analysis and analyzed using a standard curve over a concentration range of 0.02-2.0 mg/mL which had a power regression fit of R²=0.999. C2E5 non-aqueous gel samples stored at 4° C. for 8 weeks were monitored for C2E5 degradation using this HPLC method.

Subsequent physical characterization evaluations such as differential scanning calorimetry, scanning electron microscope imaging, rheological measurement and pharmacokinetic studies were performed only with the formulations containing the EC 10 polymer due to the fact that formulations containing EC10 polymer demonstrated better stability profile over those formulations containing the EC7 and EC 100 polymers and the EC10 polymer was determined to be the gelling agent of choice for this non-aqueous gel matrix.

### Results

Table 33 summarizes the 12 formulations prepared by the direct mixing method and the results from the phase separation and uniformity tests after a 3 month storage under accelerated storage conditions (40±2° C. and 75±5% relative humidity). During the storage period, phase separation was observed in the formulations with EC7 and EC 10 contents lower than 10% w/w. The C2E5 gel formulations prepared by the direct mixing method appeared to be opaque and exhibited low content homogeneity. This was especially noticeable for those gels formulated with EC100 which has longer polymer chains and a higher molecular weight. Unexpectedly high C2E5 concentrations in two of the four EC100 formulations (Formulation F9 and F12) were observed, which may be the result of insufficient solubilization of EC100 in C2E5 and MIGLYOL® 840. In general, the C2E5 non-aqueous gels prepared using the direct mixing method failed to meet the quality standard for pharmaceutical applications.

TABLE 33
Formulations prepared using the direct mixing method
and related physical characterization data.

| For- | | | | | | | |
|---|---|---|---|---|---|---|---|
| mula- | | | MIGLYOL ® | | Gel | Uni- | % C2E5 |
| tion | EC | EC | 840 | C2E5 | appear- | for- | remain- |
| Code | Type | (%) | (%) | (%) | ance | mity | ing* |
| F1 | EC7 | 7 | 63 | 30 | N/A^{#} | Low | 91.5 |
| F2 | EC7 | 8 | 72 | 20 | N/A^{#} | Low | 88.7 |
| F3 | EC7 | 14 | 56 | 30 | Opaque | Low | 85.3 |
| F4 | EC7 | 16 | 64 | 20 | Opaque | Low | 85.8 |
| F5 | EC10 | 7 | 63 | 30 | N/A^{#} | Low | 90.0 |
| F6 | EC10 | 8 | 72 | 20 | N/A^{#} | Low | 86.2 |
| F7 | EC10 | 10 | 60 | 30 | Opaque | Low | 87.1 |
| F8 | EC10 | 12 | 68 | 20 | Opaque | Low | 88.2 |
| F9 | EC100 | 7 | 63 | 30 | Opaque | Low | 104.3 |
| F10 | EC100 | 8 | 72 | 20 | Opaque | Low | 88.2 |
| F11 | EC100 | 10 | 60 | 30 | Opaque | Low | 81.6 |
| F12 | EC100 | 12 | 68 | 20 | Opaque | Low | 108.9 |

*Stability after storage at 40 ± 2° C. and 75 ± 5% relative humidity for three months;
N/A: Not applicable;
^{#}Phase separation.

The solvent evaporation method was subsequently used to prepare the C2E5 non-aqueous gel formulations. To maximize C2E5 loading in the gel matrix and achieve desirable rheological and mechanical properties, a formulation that contained 40% C2E5, 20% EC10 and 40% MIGLYOL® 840 (Formulation F13) was prepared and yielded a slightly yellow translucent gel which was determined to have a density of 1.02 g/cm³. There were no C2E5 degradation products detected by HPLC for samples stored at 4° C. for 8 weeks, and there was no statistical difference between the C2E5 concentrations in freshly prepared samples versus stored samples.

The C2E5 non-aqueous gels prepared by the direct mixing method failed to yield a homogenous gel product that meets the stringent quality standard requirements for pharmaceuticals. An optimized procedure using the solvent evaporation method produced C2E5 non-aqueous gels that were clear and homogenous. This method was successfully scaled up to prepare 400 g of C2E5 non-aqueous gels in one batch. In addition, the solvent evaporation method was also used to manufacture uniform C2E5 non-aqueous formulations of with EC concentrations as high as 20%, something not achievable by the direct mixing method. The solvent evaporation method, in which EC10 was completely solubilized in anhydrous ethanol, eliminated the problems related to residual EC particulates encountered when using the direct mixing method (Heng et al. “Development of Novel Nonaqueous Ethylcellulose Gel Matrices: Rheological and Mechanical Characterization” Pharmaceutical Research 2005; 22(4):676-84). Homogeneous C2E5 non-aqueous gels were successfully prepared, as confirmed by DSC and SEM, and used in pharmacokinetic and radionuclide decorporation efficacy studies. While not wishing to be bound to any particular theory, it is believed that any residual ethanol present in the gel system would not interact with the C2E5 molecules and might retard its hydrolysis.

### Example 7

### In Vitro Release of C2E5 Non-Aqueous Gels

### Methods

An in vitro release study was carried out using a vertical diffusion cell system equipped with an autosampler (Hanson Microette Autosampling System, Hanson Research Co., USA) to evaluate the non-aqueous gels with a formulation of 20% of EC 10, 40% of C2E5 and 40% of MIGLYOL® 840 prepared using the solvent evaporation method. The area for permeation was 1.767 cm², and the receiver compartment volume was 7 mL. The experiments were run at 32° C. and stirred magnetically at 400 rpm. The receiver medium was 0.1M phosphate buffer (pH 7.4). The cellulose acetate membrane (25 mm in diameter, 0.45 μm in diameter, Whatman®) was first treated by soaking in the receiving medium and then mounted and clamped between the receiver and donor compartments of the diffusion cells. Approximately 300 mg of C2E5 non-aqueous gel samples were loaded evenly on the surface of the cellulose acetate membrane (n=5), followed by covering the dosage form with a glass disk to occlude samples from air exposure. One mL samples were removed at 0.5 h, 1 h, 2 h, 4 h, and 6 h and replaced with fresh receiving medium.

C2E5 content in collected samples was determined by the HPLC method described in Example 6. The cumulative amount of C2E5 released per unit membrane area from the tested non-aqueous gel was plotted as a function of square root of time and as a function of time. Higuchi equation (Equation 1) dictates the drug release from semisolid dosage forms including cream, gel and ointment, which holds true when the released drug from the vehicle is below 30% (Higuchi T. Physical chemical analysis of percutaneous absorption process from creams and ointments. J Soc Cosmet Chem. 1960; 11(2):85-97. (Higuchi W. “Analysis of data on the medicament release from ointments” Journal of Pharmaceutical Sciences 1962; 51(8):802-4.):

$$Q = 2 {C}_{veh} \sqrt{\frac{Dt}{π}} , \qquad ( Equation 1 )$$

where Q=amount of drug released per unit area (mg/cm²); Cᵥₑₕ=initial drug concentration (mg/cm³) in the vehicle; D=apparent diffusion coefficient (cm²/h); t=time (h); π=constant. The release rate constant k of the C2E5 from the non-aqueous gel formulation was determined using a simplified form of Higuchi equation. The Equation 1 could be simplified as set forth Equation 2 below:

Q=k\sqrt{t} (Equation 2),

where k is the release rate constant which is determined from the slope of the cumulative amount of C2E5 released per unit membrane area from the tested non-aqueous gel versus square root of time.

Fick's law (Equation 3) has been used as a simple model to describe the steady-state diffusion of drug through the synthetic membranes and skins:

$${J}_{ss} = ( \frac{D * {K}_{p}}{h} ) * A * {C}_{veh} , \qquad ( Equation 3 )$$

where Jₛₛ=steady-state flux (mg/h); D=drug diffusivity (cm²/h); h=membrane thickness (cm); Kₚ=drug's membrane-vehicle partition coefficient; Cᵥₑₕ=initial drug concentration (mg/cm³) in the vehicle; and A=surface area (cm²). Jₛₛ can be determined from the slope of the linear plot in the steady-state region of the cumulative amount of C2E5 permeated (mg) per unit diffusion surface (cm²) versus a function of time.

For the duration of this experiment, the maximum C2E5 concentration detected in the receptor compartment at 6 h only reached about 20% to 30% of the solubility of C2E5 at pH 7.4, which is 2.2 mg/mL (Sueda et al. “Physicochemical characterization of a prodrug of a radionuclide decorporation agent for oral delivery” J Pharm Sci. 2012 May 29). A steady-state flux condition and sink condition were maintained for the duration of the experiment.

### Results

Table 34 lists the cumulative amounts of C2E5 released, release rate constant k, steady-state flux Jₛₛ per unit area calculated from the 5 individual in vitro release runs and their respective average ±SD of the in vitro release study of the non-aqueous gel of 20% EC10, 40% C2E5 and 40% MIGLYOL® 840 (Formulation F13).

The cumulative amount of C2E5 released per unit membrane area after 6 h from the 5 individual in vitro release runs ranged from 2.890 mg/cm² to 3.3326 mg/cm², with an average value at 3.108±0.170 mg/cm², which has a coefficient of variance of 5.5%. The average release rate constant k and average steady-state flux Jₛₛ per unit area of the 40% C2E5 non-aqueous gel were determined to be 1.572±0.088 mg/cm²/h^{0.5} and 0.556±0.031 mg/cm²/h, respectively, which both have a coefficient of variance of 5.6%. For each individual run, the linear regression fit of R²≧0.994 and R²≧0.969 were achieved for release rate constant k and average steady-state flux Jₛₛ per unit area, respectively.

TABLE 34
Cumulative amounts of C2E5 released, release rate constant k, steady-state flux Jₛₛ per unit area calculated
from the 5 individual in vitro release runs and their respective average ± SD of the in vitro release
study of the non-aqueous gel of 20% EC10, 40% C2E5 and 40% MIGLYOL ® 840 (Formulation F13).

| | Cumulative | Average Cumulative | | | | |
|---|---|---|---|---|---|---|
| | Amount of C2E5 | Amount of C2E5 | | | Steady-state | Average Steady-state |
| | Released | Released | Release Rate | Average | Flux Jₛₛ per | Flux Jₛₛ per |
| | After 6 h | After 6 h ± SD | Constant k | Flux k ± SD | unit area | unit area |
| Run | (mg/cm²) | (mg/cm²) | (mg/cm²/h^{0.5}) | (mg/cm²/h^{0.5}) | (mg/cm²/h) | (mg/cm²/h) |
| 1 | 3.202 | 3.108 ± 0.170 | 1.601 | 1.572 ± 0.088 | 0.568 | 0.556 ± 0.031 |
| 2 | 3.326 | | 1.684 | | 0.600 | |
| 3 | 3.003 | | 1.515 | | 0.538 | |
| 4 | 2.890 | | 1.457 | | 0.518 | |
| 5 | 3.119 | | 1.604 | | 0.556 | |

The in vitro release testing has become the most widely used test for semisolid products to monitor the release of the drug from the matrix, test product content uniformity, and compare batch to batch performance after storage and changes in manufacturing process (Shah et al. “Release of Hydrocortisone from Topical Preparations and Automated Procedure” Pharmaceutical Research 1991; 8(1):55-9 and Vinod S. “In Vitro Release from Semisolid Dosage Forms? What Is Its Value?” Percutaneous Absorption: Informa Healthcare; 2005. p. 481-8). Although the delivery of C2E5 transdermally into systemic circulation is a multistep process, the in vitro release results suggest that the C2E5 is readily to be released from the non-aqueous gel matrix and possess suitable steady-state flux Jₛₛ to deliver sufficient C2E5 for decorporation in vivo. The narrow distribution of release rate constant k with a coefficient of variance of 5.6% confirmed the optimal content uniformity of the 40% C2E5 non-aqueous gel prepared by solvent evaporation method.

### Example 8

### In Vitro Percutaneous Permeation of C2E5

### Methods

An in vitro permeation study was carried out using a vertical diffusion cell system equipped with an autosampler (Hanson Microette Autosampling System, Hanson Research Co., USA). The area for permeation was 1.767 cm², and the receiver compartment volume was 7 mL. The experiments were run at 32° C. and stirred magnetically at 400 rpm. The receiver medium was 0.1M phosphate buffer (pH 7.4). SD rat skin samples were prepared by harvesting dorsal skin samples from freshly euthanized (<1 h) SD rats (n=9, 200-300 g). The skin was shaved and the subcutaneous fat tissue was carefully peeled from the dermis layer to remove potential interference due to high levels of esterase activity in subcutaneous tissue. The processed skin samples were inspected visually for membrane integrity. The qualified skin samples were stored at −20° C. until use. The SD rat skin samples were first allowed to thaw for at least 1 h in the receiver medium, and then mounted and clamped between the receiver and donor compartments of the diffusion cells. 100 μL of C2E5 nonaqueous gel samples containing 6% EC10, 25% C2E5 and 69% MIGLYOL® 840 that were spiked with [14C]-C2E5 were loaded evenly onto the surface of the rat skin (n=3′), and 1.0 mL samples were removed at each hour from time 0 to 23 h and replaced with fresh receiving medium. ([¹⁴C]-C2E5; 50 mCi/mmol) labeled at carbon-1 (carbonyl carbon) was purchased from American Radiolabeled Chemicals, Inc., St. Louis, Mo.).

Collected samples were dispensed into Ultima Gold™ scintillation cocktail at a ratio of 100 μL:10 mL and assayed directly for radioactivity by liquid scintillation counting (LSC) using a Packard TriCarb 3100TR (PerkinElmer Life and Analytical Sciences) with automatic quench correction. Samples were counted for 10 min or until a 5% confidence level was achieved. The samples were subsequently assayed for intact C2E5 and its metabolites using HPLC-FSA. The cumulative releases of C2E5 and its metabolites were plotted as a function of time.

### Results

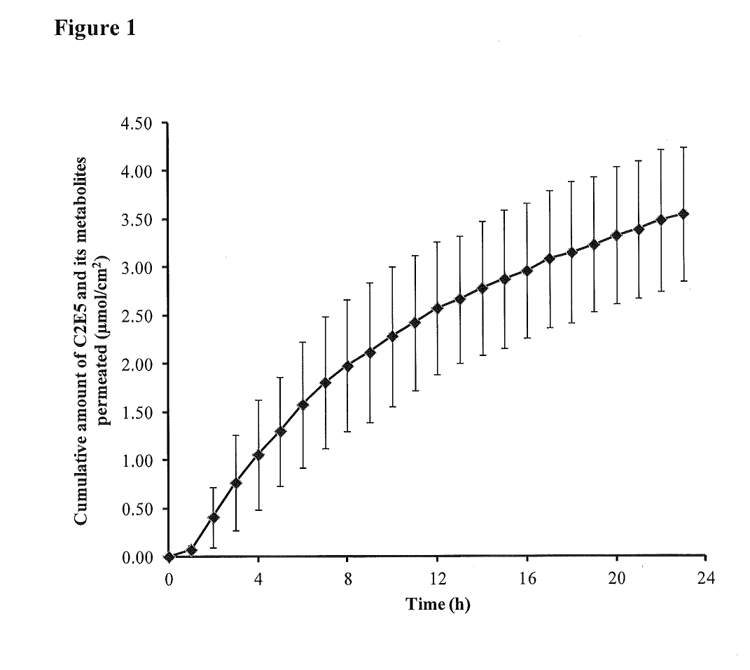

Fick's law (Equation 4) has been used as a simple model to describe the steady-state diffusion of drug through the skin:

$${J}_{ss} = ( \frac{D * {K}_{\frac{sc}{veh}}}{h} ) * A * {C}_{veh} , \qquad ( Equation 4 )$$

where Jₛₛ=steady-state flux (mol/hr); D=drug diffusivity (cm²/hr); h=membrane thickness (cm); K_{sc/veh}=drug's stratum corneum-vehicle partition coefficient; Cᵥₑₕ=drug concentration (mg/cm³) in the vehicle; and A=surface area (cm²). The cumulative amount of C2E5 and its metabolites permeated through the SD rat skin versus time is presented in FIG. 1, The overall concentrations of C2E5 and its metabolites in receiver compartment at different time points were calculated based on Equation 5.

$$Conc {.}_{C 2 E 5 and its metabolites} = Conc {.}_{[ {}^{14} C ] C 2 E 5 and its metabolites} \times \frac{Conc {.}_{total C 2 E 5 i n gel}}{Conc {.}_{[ {}^{14} C ] C 2 E 5 i n gel}} \qquad ( Equation 5 )$$

The permeation profile in FIG. 1 exhibits a plateau effect starting from the 8 h sample as a result of C2E5 depletion in the donor compartment. Calculation of the cumulative amount of C2E5 and its metabolites permeated at 8 h revealed that ˜10% of the initially applied C2E5 dose had permeated through the rat skin and into the receiver compartment, which became a finite dosing scenario due to >10% of bioavailability of the C2E5 [19]. Assuming that the C2E5 permeation reached a steady-state flux between 2 h and 7 h, the Jₛₛ per unit area and the diffusional lag time of C2E5 through SD rat skin can be calculated from FIG. 1 to be 0.276 μmol/cm²/h and 0.33 h, respectively (y=0.2758x−0.0899, R²=0.995).

The concentrations of C2E5 and its metabolites in the collected samples from the receiver compartment at different time points were analyzed by HPLC-FSA. The concentrations of DTPA in receiver compartment at different time points were calculated based on Equation 6.

$$Conc {.}_{DTPA} = Conc {.}_{C 2 E 5 and its metabolites} \times \frac{Conc {.}_{[ {}^{14} C ] DTPA detected i n sample}}{Conc {.}_{total [ {}^{14} C ] radio activity detected i n sample}} \qquad ( Equation 6 )$$

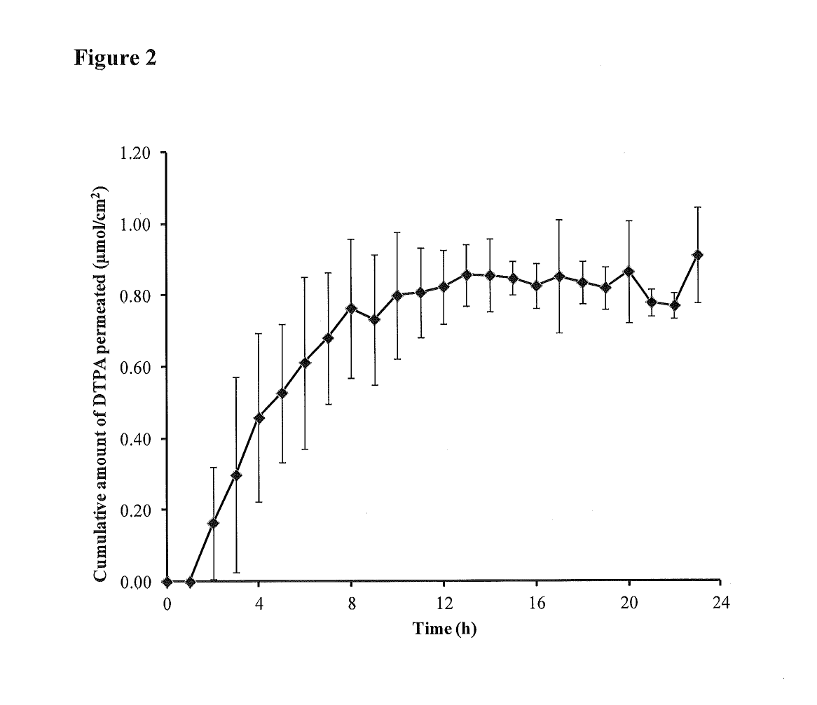

The radiochromatograms of the samples revealed a pronounced DTPA presence in the collected receiver compartment samples, representing from 17% to 50% (primarily 30% to 40%) of the injected radioactivity ranging from 2 h to 23 h. The cumulative amount of DTPA permeated through the SD rat skin versus time is presented in FIG. 2. The cumulative amount of DTPA reached a plateau at 0.75 to 0.80 μmol/cm² after 8 h, which could be indicative of loss of esterase activity in the rat skin. However, it was encouraging to observe that C2E5 was converted to DTPA by esterases in the rat skin which was only minimally observed in the in vitro C2E5 metabolism studies using SD rat skin S9 fractions. Without wishing to be limited to any particular theory, considering that the gradual increase in the cumulative amount of C2E5 and its metabolites that permeated through the rat skin (1.98 μmol/cm² at 8 h to 3.55 μmol/cm² at 23 h), it may be concluded that this is primarily the result of the accumulation of the C2E 1 to C2E5 species in the receiver compartment. An increase in the amounts of C2E4 and C2E5 accumulating in the receiver chamber from 8 h to 23 h was confirmed by HPLC-FSA. During the passive diffusion process of drugs through skin, it is generally accepted that small hydrophilic molecules are rate controlled by the stratum corneum, while small lipophilic molecules are rate controlled by their partition from the stratum corneum into the aqueous epidermis [16]. Because the enzymatic conversion of C2E5 to DTPA by the esterases in the epidermis results in change in partition coefficient of over 8 orders of magnitude, the esterase activity in the epidermis maintains the flux of C2E5 and its metabolites as they traverse the skin.

With the key parameters obtained from the in vitro percutaneous permeation study of C2E5 nonaqueous gel through rat skin, the dose of the C2E5 gel for the in vivo decorporation study was estimated using relevant pharmacokinetic data from previous studies. The 18.5 min half-life of DTPA in extracellular fluid reported by Crawley and Haines, and the volume of distribution derived from the method used by Volf (assumes 6.5 mL blood per 100 g body weight and plasma volume equals 55 percent of the whole blood volume) were used to calculate the in vivo clearance of DTPA in rats (Crawley and Haines, “The dosimetry of carbon-14 labelled compounds: the metabolism of diethylenetriamine pentaacetic acid (DTPA) in the rat,” Int J Nucl Med Biol, 6 (1979) 9-15 and Volf “Chelation therapy of incorporated plutonium-238 and americium-241: comparison of LICAM(C), DTPA and DFOA in rats, hamsters and mice,” Int J Radiat Biol Relat Stud Phys Chem Med, 49 (1986) 449-462). For a 250 g rat, the clearance of DTPA was calculated to be 0.02 L/h. For the steady state concentration of DTPA (Css_{DTPA}) obtained from zero-order release of transdermal delivery of C2E5 gel, a concentration of 25 μM was assumed based on data reported by Ansoborlo and coworkers (Ansoborlo et al. “Review of actinide decorporation with chelating agents,” Cr Chim, 10 (2007) 1010-1019). The rate of input of DTPA needed for maintaining its concentration for effective decorporation was determined to be 0.5 μmol/h. With a Jₛₛ per unit area of 0.276 μmol/cm²/h calculated from FIG. 1 and the conservative assumption of 20% of C2E5 converted to DTPA by skin esterases, the required application area of the C2E5 gel was calculated to be 9.06 cm². Because the C2E5 concentration in the C2E5 nonaqueous gel used in the in vivo decorporation study and the in vitro percutaneous permeation study were 40% and 25%, respectively, the application area for the in vivo decorporation study was adjusted to 6.0 cm² based on Fick's law. To determine the C2E5 dose for the in vivo decorporation study, an approach to maintain an infinite dosing regimen for the first 24 h was adopted for optimal decorporation based on the biokinetic profile of the wound contamination model by [²⁴¹Am]-Americium nitrate [12]. Considering the Jₛₛ per unit area derived from FIG. 2 and that the potential loss of esterase activity in the rat skin might lead to a lower flux value, a C2E5 dose of 1000 mg/kg was selected for application to the contaminated rats. This was believed to result in a 13.6% of applied C2E5 being delivered in the first 24 h under an ideal scenario. In reality, it was believed to reach less than 10% of delivery rate for applied C2E5 for the first 24 h period until the residual C2E5 gel removal due to potential reduction and exhaustion of skin esterases' hydrolytic capability.

### Example 9

### Pharmacokinetics of Neat C2E5 and C2E5 Non-Aqueous Gel

### Methods

All animal studies were conducted according to a protocol approved by the University of North Carolina at Chapel Hill Institutional Animal Care and Use Committee (IACUC). Ten-week-old adult female Sprague-Dawley (SD) rats weighing from 200 to 300 g were used in these studies (Charles River Labs, Raleigh, N.C.). Food and water were given ad libitum. The animal room was kept at a controlled temperature (23° C.) and light cycle (light exposure from 8 AM to 8 PM). For the duration of the study, the rats were housed in metabolic cages individually with urine and feces collection until euthanasia at 24 h after neat C2E5 or C2E5 gel application.

The dorsal skin between the cervical vertebrae and anterior thoracic vertebrae of SD rats was carefully clipped followed by application of a C2E5 non-aqueous gel containing 20% of EC10, 40% of C2E5, and 40% of MIGLYQL® 840 at a dose of 200 mg/kg to a 2 cm×3 cm region using a cotton swap. Neat C2E5 (i.e., only C2E5) at a dose of 200 mg/kg was applied using a 1 mL syringe to the same size area as a positive control. A jacket with a plastic dorsal insert (VWR international) was placed on the rats to protect the applied treatments. Blood samples (0.4 mL) were collected into 3 mL syringes with a small volume of sodium heparin prior to treatment and at 0.5 h, 1 h, 2 h, 4 h, 8 h, 12 h and 24 h after treatment. The collected samples were immediately transferred from the syringes into pre-chilled sampling tubes containing 5 mg sodium fluoride and 4 mg potassium oxalate (BD vacutainer 367921). The tubes were inverted 8 times as per manufacturer's suggestion, pre-chilled to 4° C. and then centrifuged for 10 min at 1300×g. Plasma samples were then portioned into 1.7 mL Eppendorf tubes which contained an equal amount of a 20% formic acid aqueous solution. These tubes were immediately vortexed and stored at −20° C. until analysis. The mass of neat C2E5 or C2E5 gel applied was recorded for each animal to permit actual dose determination. The animals were transferred and housed in metabolic cages individually after C2E5 treatment. Animals were euthanized 24 h after C2E5 gel or neat C2E5 application. The animals were observed during the study period and the body weight of the animals was recorded at pre-dose and prior to necropsy.

An LC/MS/MS method was developed for the analysis of C2E5 and metabolites except for the fully de-esterified metabolite, DTPA, in these samples. Acidified plasma samples (100 μL) were first treated with 25 μL of ¹³C—C2E5 stable-label internal standard (1000 ng/mL), followed by precipitation with MeCN (400 μL). The supernatant (400 μL) was removed, evaporated to dryness and the residue was reconstituted with 500 μL of 85/15/0.1% water/MeCN/formic acid. A 10 μL injection was used for LC/MS/MS analysis. Reverse-phase chromatography was performed at 0.3 mL/min on a YMC ODS-AM C18 (100×2 mm, 3 μm) column with mobile phases A (MPA, 0.1% formic acid in water) and B (MPB, 0.1% formic acid in MeCN) using a 10 min gradient (isocratic at 13% MPB for 1 min, linear gradient to 50% MPB at 6 min, linear gradient to 60% MPB at 6.5 min, linear gradient to 90% MPB at 8 min, return to initial 13% MPB at 8.1 min, and equilibrate at 13% MPB until 10 min). After separation by liquid chromatography, the analytes and internal standard were detected on a triple quadrupole mass spectrometer using heated electrospray ionization (HESI-II) in the positive-ion mode.

For detection of DTPA, acidified plasma samples (100 μL) were first treated with 50 μL of 2 mM iron (III) chloride hexahydrate, followed by addition of 400 μl, of ¹³C-DTPA stable-label internal standard (100 ng/mL in 0.1% acetic acid in MeCN). This solution was vortexed for 5 min and then centrifuged at 3,000 rpm for 10 min. The supernatant (350 μL) was removed, evaporated to dryness and reconstituted with 100 μL, of 0.1% aqueous acetic acid. A 10 μL aliquot of the reconstituted sample was used for LC/MS/MS analysis. The highly polar nature of DTPA required the incorporation of ion-pairing chromatography to produce acceptable LC peak shape and retention. Mobile phase A (MPA) was 90:10 water:methanol with 1 mM ammonium formate and 1 mM tributylamine and mobile phase B (MPB) was 50:50 MeCN:(5:95 water:methanol) with 1 mM acetic acid and 1 mM tributylamine. A 10 min gradient was used to afford separation (isocratic at 5% MPB for 1 min, linear gradient to 80% MPB at 7 min, linear gradient to 90% MPB at 7.1 min, maintaining at 90% MPB through 8 min, return to initial 5% MPB at 8.1 min, and equilibrate at 5% MPB until 10 min). Reverse-phase chromatography was performed at 0.3 mL/min on an Advanced Materials Technology HALO® Phenyl-Hexyl column (50×2.1 mm, 2.7 μm). The analyte and internal standard were detected on a triple quadrupole mass spectrometer using heated electrospray ionization (HESI-II) in the negative-ion mode.

### Results

The concentrations of C2E5 and its partially hydrolyzed metabolites, DTPA tetra-ethyl esters (C2E4), DTPA tri-ethyl esters (C2E3), DTPA di-ethyl esters (C2E2), DTPA mono-ethyl esters (C2E1), and the fully hydrolyzed metabolite, DTPA in samples obtained at selected times points after application of neat C2E5 or a 40% C2E5 non-aqueous gel (200 mg/kg) are listed in Table 35. C2E5 and C2E4 were detected in only a small number of samples in both neat C2E5 and 40% C2E5 non-aqueous gel treatment groups. On the other hand, C2E3 and C2E2 consistently appeared in plasma samples throughout the sampling period from very early time points through the end of the 24 h sampling period. The Cₘₐₓ of C2E3 reached 208 ng/mL at 8 h and 155 ng/mL at 0.5 h for 40% non-aqueous gel group and neat C2E5 group, respectively. The Cₘₐₓ of C2E2 reached 175 ng/mL at 4 h and 45 ng/mL at 12 h for 40% non-aqueous gel group and neat C2E5 group, respectively. The 40% C2E5 non-aqueous gel apparently delivered C2E3 and C2E2 in a more consistent and sustained manner than observed from the neat C2E5 treatment group. This indicates that a zero order release of C2E5 from the gel matrix was achieved. It clearly demonstrated an advantage of C2E5 gel formulation over neat C2E5 formulation for transdermal delivery. Due to the runny nature of the neat C2E5, it was difficult to accurately apply neat C2E5 to the shaved dorsal skin and keep it at the application site for a prolonged period, which is a prerequisite to achieve sustained delivery of any drug via skin. C2E1 was not detected in plasma samples from either the neat C2E5 or the C2E5 non-aqueous gel treatment groups. Some DTPA was detected in samples from both treatment groups obtained 8 hours or later, indicating that C2E5 may indeed be a prodrug of DTPA. The areas under curve (AUC) determinations for all of the C2E5 metabolites appear in Table 36. The AUCs of C2E3, C2E2 and DTPA obtained from the neat C2E5 treatment group appeared to be smaller than those obtained from the 40% C2E5 non-aqueous gel treatment group.

TABLE 35
Concentrations of C2E5 and its metabolites in SD rat plasma samples at various time points
after application of neat C2E5 or 40% C2E5 non-aqueous gel at 200 mg/kg dose level (n = 4).

| | C2E4 conc.* | | C2E3 conc.** | | C2E2 conc. *** | | DTP A conc. **** | |
|---|---|---|---|---|---|---|---|---|
| | (ng/mL) | | (ng/mL) | | (ng/mL) | | (ng/mL) | |
| Sample | Neat | C2E5 | Neat | C2E5 | Neat | C2E5 | Neat | C2E5 |
| Time (h) | C2E5 | Gel | C2E5 | Gel | C2E5 | Gel | C2E5 | Gel |
| 0 | ND^{a} | ND^{b} | 4.1 ± 3.7^{b} | 1.9^{a} | ND^{b} | ND^{b} | ND^{b} | ND^{b} |
| 0.5 | 13.5^{a} | ND^{a} | 155.2 ± 164.3^{b} | 54.1^{a} | ND^{b} | 26.2^{a} | 14.7^{a} | ND^{a} |
| 1 | ND^{a} | 59.9^{a} | 20.3 ± 8.1^{c} | 202.6 ± 116.3^{c} | ND^{c} | 62.5 ± 36.7^{c} | ND^{c} | ND^{c} |
| 2 | 52.7^{a} | 83.8^{a} | 92.0 ± 112.7 | 173.4 ± 129.1 | 14.1 ± 1.3^{b} | 134.1 ± 83.3 | ND | ND |
| 4 | ND^{c} | ND | 52.5 ± 15.2^{c} | 161.1 ± 105.3 | 13.1 ± 3.4^{c} | 174.8 ± 164.4 | ND^{c} | ND |
| 8 | ND | ND | 27.3 ± 9.2 | 208.7 ± 187.1 | 14.4 ± 2.7^{c} | 130.8 ± 123.2 | 17.6 ± 9.9^{b} | 11.2 ± 0.4^{b} |
| 12 | ND | ND | 52.6 ± 38.6 | 120.5 ± 76.0 | 44.5 ± 47.6 | 84,7 ± 56.9 | 13.2^{a} | 692.9^{a} |
| 24 | 45.1^{a} | ND | 56.6 ± 21.9 | 132.1 ± 68.1 | 35.0 ± 19.3 | 126.0 ± 76.4 | 46.1 ± 21.6^{b} | 12.0^{a} |

*Detection limit: 10 ng/mL;
**Detection limit: 1.0 ng/mL;
*** Detection limit: 10 ng/mL;
**** Detection limit: 10 ng/mL;
ND: Not Detected;
^{a}one sample;
^{b}two samples;
^{c}three samples.

TABLE 36
Area under curve (AUC) for C2E5 metabolite species in pharmacokinetic
study after application of neat C2E5 or 40% C2E5 non-aqueous
gel at 200 mg/kg dose level and ratio of AUC of C2E5 metabolite
species from neat C2E5 group over AUC of C2E5 metabolite species
from 40% C2E5 non-aqueous gel group.

| | AUC_{0-24 h} | | |
|---|---|---|---|
| Metabolites | (h* ng/mL) | | AUC_{0-24 h (C2E5 gel)}/ |
| species | Neat C2E5 | C2E5 gel | AUC_{0-24 h (Neat C2E5)} |
| C2E4 | 118.9 ± 136.4 | 113.7 ± 76.1 | 0.96 |
| C2E3 | 1205.8 ± 158.6 | 3522.7 ± 2258.5 | 2.92 |
| C2E2 | 660.3 ± 350.7 | 2747.7 ± 1888.8 | 4.16 |
| DTPA | 208.0 ± 227.3 | 2852.5 ± 3867.3 | 13.71 |

### Example 10

### Radionuclide Decorporation of Contaminated Rats

### Methods

All animal studies were conducted according to a protocol approved by the University of North Carolina at Chapel Hill Institutional Animal Care and Use Committee (IACUC). Ten-week-old adult female SD rats weighing from 200 to 300 g were used in these studies (Charles River Labs, Raleigh, N.C.). Food and water were given ad libitum. The animal room was kept at a controlled temperature (23° C.) and light cycle (light exposure from 8 AM to 8 PM). For the duration of the study, the rats were housed in metabolic cages individually with daily urine and feces collection until euthanasia on Day 7.

To evaluate the efficacy of transdermal delivery of the C2E5 nonaqueous gel, a radionuclide decorporation efficacy study was conducted in rats contaminated with ²⁴¹Am. Adult female SD rats were anesthetized with 2-3% isoflurane. Dorsal skin between the cervical vertebrae and anterior thoracic vertebrae was clipped with caution before all animals were contaminated with [²⁴¹Am]-Americium nitrate solution (250 nCi, 0.1 mL) via an i.m. injection in anterior thigh muscle. The C2E5 nonaqueous gel was applied at dose of 1000 mg/kg using a cotton swap to a 6 cm² (2 cm×3 cm) area of the clipped dorsal region immediately after contamination. A jacket with plastic dorsal insert (VWR International) was placed on the rat to protect the gel application region. The mass of C2E5 gel applied was recorded for each animal to permit the actual dose determination. The animals were housed in metabolic cages individually after C2E5 treatment and were euthanized 7 days after contamination. Positive and negative controls included animals administered Ca-DTPA intravenously and animals without any treatment. The animals were observed once daily and their body weights recorded at pre-dose and prior to necropsy, Urine and feces were collected daily until euthanasia, when the liver, kidneys, both femurs and the muscle tissue from around the injection site were also collected. Cage washes were collected at the end of each experiment. As ˜35% of the decay of ²⁴¹Am is associated with photon emissions of 59.7 keV, ²⁴¹Am present in samples was quantified using a gamma counter (2470 Wizard 2, Perkin Elmer). The samples were counted for one minute using a 40-80 keV energy detection window and were background-corrected. Additionally, ²⁴¹Am activity was quantified in 2×0.1 mL aliquots of the dosing solution. For all samples, ²⁴¹Am content was expressed as a percentage of the initial dose. The percent of decorporation enhancement and reduction in organ burden for animals treated with i.v. Ca-DTPA or transdermal C2E5 gel compared to the control animals were calculated from Equation 7 (Guilmette et al., “Toward an optimal DTPA therapy for decorporation of actinides: time-dose relationships for plutonium in the dog” I. Radiat Res, 78 (1979) 415-428):

$$Percentage change = \frac{\vert \begin{matrix}\% ID ( Control group ) - \\ \% ID ( Treament group )\end{matrix} \vert}{\% ID ( Control group )} , \qquad ( Equation 7 )$$

where % ID represents the percent of injected dose. Where data for both femurs were available, an estimate of the total skeletal burden was made using the method of Volf with modification in which the counts in the contra-lateral femur were multiplied by 19 and added to the counts in the ipsi-lateral femur. The modification for the literature method was due to higher ²⁴¹Am content in ipsi-lateral femur as a result of i.m. injection in anterior thigh muscle.

Statistically significant differences among groups were determined using one-way ANOVA with Tukey's post hoc comparison. All measurements are expressed as mean±standard deviation (S.D.). The level of significance was set at p<0.05.

### Results

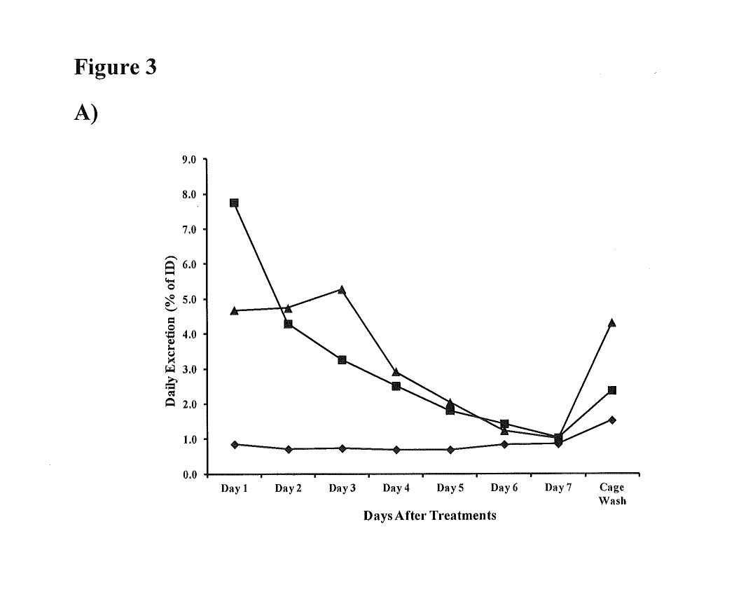

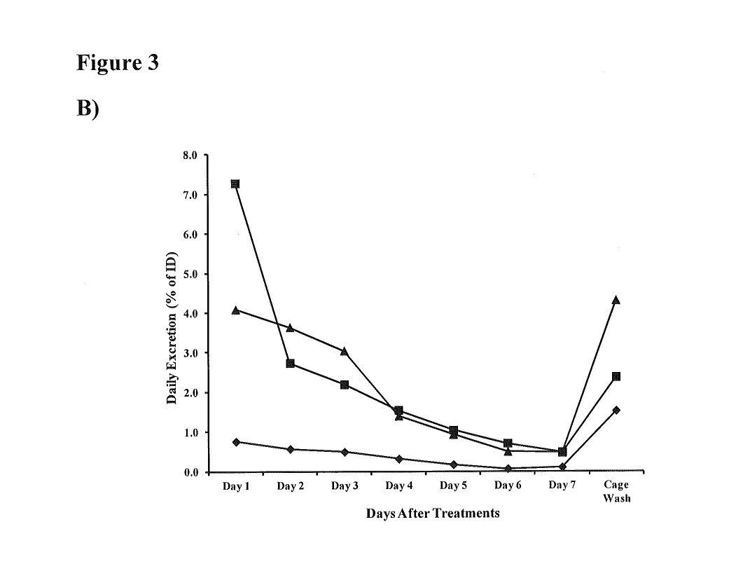

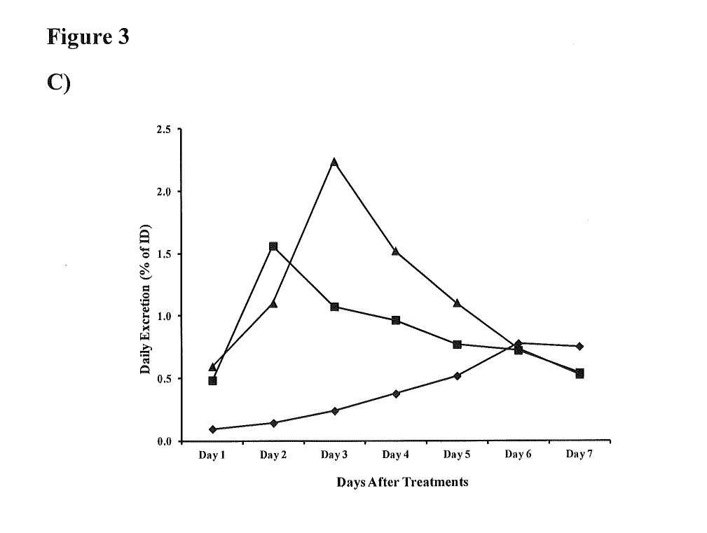

Table 37 summarizes the excretion of ²⁴¹Am in rats and the residual tissue content after seven days following a single dose treatment with the C2E5 nonaqueous gel. The cumulative amounts of ²⁴¹Am collected in urine and feces as well as the reduction in liver and skeleton burden are presented in Tables 38 and 39, respectively. In the wound contamination model, it has been reported that ²⁴¹Am at the intramuscular contamination site initially enters the bloodstream as the stable trivalent ²⁴¹Am³⁺ form. Approximately 95% of the ²⁴¹Am³⁺ is cleared from the plasma in less than 1 h with the majority accumulating in the liver and skeleton (Menetrier et al. “The biokinetics and radiotoxicology of curium: a comparison with americium” Appl Radiat Isot, 66 (2008) 632-647). If there is adequate concentration of chelators in the blood to sequester ²⁴¹Am³⁺ in the form of a stable chelation complex, the translocation and deposition of radionuclides into the adjacent bone and other tissues can be greatly reduced, if not totally prevented. In contrast to plutonium, which is almost 100% protein bound in plasma, only 30% of ²⁴¹Am³⁺ in the circulation is protein bound (Ansoborlo et al.), which makes ²⁴¹Am³⁺ more readily available to interact with chelators in the bloodstream. A significant increase in the elimination of ²⁴¹Am in the urine and feces was observed in rats following topical administration of the C2E5 nonaqueous gel compared to the no-treatment control group (Table 38). The decorporation efficacy of the transdermal C2E5 treatment (1000 mg/kg) was comparable to that observed for the injectable product (Ca-DTPA) when administered at standard recommended doses (14 mg/kg). The enhanced decorporation of ²⁴¹Am for i.v. Ca-DTPA and transdermal C2E5 treatment groups over the control group was 255.3% and 279.9%, respectively. FIG. 3 shows the total daily urinary+fecal excretion (FIG. 3A) as well as the daily excretion by urine (FIG. 3B) and feces (FIG. 3C) of ²⁴¹Am individually in rats after seven days following a single dose treatment of the C2E5 nonaqueous gel. The decorporation profile of i.v. Ca-DTPA showed that ²⁴¹Am excretion reached its peak in the first 24 h after administration with a ratio of urine excretion over fecal excretion at 15:1 with a rapid decline after the first day. In contrast to the i.v. Ca-DTPA group, the decorporation profile of rats treated with the C2E5 transdermal gel showed enhanced excretion of ²⁴¹Am over the control group during the first 24 h and that excretion increased over the next 24 h, peaking after 3 days. The decorporation profile of the C2E5 transdermal gel not only demonstrated that an enhanced decorporation of ²⁴¹Am for extended duration had been accomplished, but also indicated that a sustained release of DTPA and other C2E5 metabolites capable of chelating ²⁴¹Am via transdermal delivery of C2E5 had been achieved.

The ²⁴¹Am burden in the ipsi-lateral femur of the C2E5 transdermal treatment group showed statistically significant reduction over the no-treatment control. Only the C2E5 transdermal treatment group showed a statistically significant reduction in the skeleton burden of ²⁴¹Am over the control group (Table 39). In addition, the liver burdens of ²⁴¹Am in rats of i.v. Ca-DTPA and transdermal C2E5 treatment groups were significantly lower than that of the control group with a 38.2% and a 49.1% reduction, respectively (Table 39). The percent of retention of ²⁴¹Am at wound site for all experimental groups was consistent with previous literature reports (National Council on Radiation Protection and Measurements., National Council on Radiation Protection and Measurements. Scientific Committee 57-17 on Radionuclide Dosimetry Model for Wounds., Development of a biokinetic model for radionuclide-contaminated wounds and procedures for their assessment, dosimetry, and treatment: recommendations of the National Council on Radiation Protection and Measurements, Dec. 14, 2006, National Council on Radiation Protection and Measurements, Bethesda, Md., 2007 pp. 35-117). The in vivo decorporation results confirmed the validity of maintaining a concentration of 10 to 25 μM of chelators, such as DTPA, for an adequate duration to ensure optimal in vivo chelation of transuranic radionuclides (Ansoborlo et al.). The results presented here also supported the hypothesis that the mismatch of the pharmacokinetic profile of i.v. DTPA injection and the slow release profile of transuranic radionuclides.

Test animals were examined daily during the experimental period for indications of disease or abnormalities including morbidity, mortality, general appearance and signs of toxic effects. The skin area for dosing was carefully inspected pre- and post-dosing after removal of the C2E5 gel and indicated that no elevated skin reddening or local skin inflammation had occurred. A comparison of body weights of test animals at pre-dosing and at sacrifice showed no statistical difference. A 250 g rat normally possesses a surface area of 348 cm² based on a formula developed by Rubner (Farriol et al., “Body surface area in Sprague-Dawley rats” Journal of Animal Physiology and Animal Nutrition, 77 (1997) 61-65). The 6.0 cm² application area is equals to 1.72% of surface area of the 250 g rat. That can be translated to an application area of 325 cm² for a 70 kg man with a 180 cm in height and a 1.89 m² body surface area, which is equals to the an area of circle with a diameter of 20.3 cm. Applying the dose multiplier 0.162 to convert a rat dose to a human dose, as listed in 2005 FDA guidance for estimating the maximum safe starting dose in initial clinical trials for therapeutics in adult healthy volunteers, it was determined that a C2E5 dose of 11.3 g of C2E5 dose per day was required for a 70 kg man (US FDA Guidance for Industry, Estimating the maximum safe starting dose in initial clinical trials for therapeutics in adult healthy volunteers, Center for Drug Evaluation and Research, USFDA, US Department of Health and Human Services, (2005)). While this is certainly much higher than most transdermal drugs, it is manageable, especially considering the extraordinary circumstances following a nuclear terrorism event or a case of accidental contamination with transuranic radionuclides.

TABLE 37
Excretion and tissue content of ²⁴¹Am in rats seven days following
a single dose treatment of the decorporation agents.

| | | | % of ID of ²⁴¹Am | | | |
|---|---|---|---|---|---|---|
| | | No. of | Total | | | Ipsi-lateral |
| Treatment | Route | animals | decorporation | Liver | Kidney | femur |
| Control | No treatment | 3 | 6.9 ± 1.7 | 16.5 ± 2.2 | 0.67 ± 0.04 | 1.71 ± 0.59 |
| Ca-DTPA | i.v. | 4 | 24.5 ± 3.5* | 10.2 ± 2.7* | 0.55 ± 0.03 | 0.88 ± 0.18^{c} |
| 14 mg/kg | | | | | | |
| C2E5 | Transdermal | 3 | 26.2 ± 2.7* | 8.4 ± 2.0* | 0.63 ± 0.07 | 0.54 ± 0.10* |
| 1000 mg/kg | | | | | | |

| | % of ID of ²⁴¹Am | | | | |
|---|---|---|---|---|---|
| | | | | Pelt at | Total recovery |
| | Contra-lateral | Muscle- | Muscle- | injection | of injected |
| Treatment | femur | ipsi | contra^{a} | site | ²⁴¹Am^{b} |
| Control | 0.77 ± 0.21 | 49.3 ± 6.0 | 0.0083 ± 0.0022 | 1.04 ± 0.56 | 91.5 ± 2.1 |
| Ca-DTPA | 0.57 ± 0.09 | 40.0 ± 8.5 | 0.0054 ± 0.0028 | 0.43 ± 0.24 | 91.1 ±1.2 |
| 14 mg/kg | | | | | |
| C2E5 | 0.48 ± 0.05 | 44.3 ± 2.0 | 0.0078 ± 0.0017 | 0.84 ± 0.66 | 90.9 ± 1.2 |
| 1000 mg/kg | | | | | |

*Significant difference by one-way ANOVA with Tukey's post hoc comparison of means, P < 0.05;
^{a}% of ID of ²⁴¹Am per gram of muscle-contra;
^{b}Total recovery of injected ²⁴¹Am was calculated based on formula: Total recovery of injected ²⁴¹Am = Total decorp. + Liver + Kidney + Ipsi-lateral femur + Contra-lateral femur × 19 + Muscle-ipsi + Muscle-contra * 45% of animal body weight at sacrifice + Pelt at injection site;
^{c}One animal in i.v. Ca-DTPA treatment group was excluded from skeleton burden calculation due to extremely high ²⁴¹Am-content (13.5% compared to 0.5 to 2.5% range for the rest of 9 tested animals) possibly as a result of deep i.v. injection which contaminated ipsi-lateral femur.

TABLE 38
Excretion of ²⁴¹Am after seven days, by urine and feces,
respectively, following a single dose treatment.

| | | | | | | % of Enhanced |
|---|---|---|---|---|---|---|
| | | | Cumulative excretion (% of ID) | | | decorporation |
| Treatment | Route | N | In urine | In feces | Total | over control |
| Control | — | 3 | 2.9 ± 1.2 | 4.0 ± 0.7 | 6.9 ± 1.7 | — |
| Ca-DTPA | i.v. | 4 | 18.3 ±2.5* | 6.1 ± 1.1* | 24.5 ± 3.5* | 255.3 |
| 14 mg/kg | | | | | | |
| C2E5 | Transdermal | 3 | 18.4 ± 1.8* | 7.8 ± 1.2* | 26.2 ± 2.7* | 279.9 |
| 1000 mg/kg | | | | | | |

One-way ANOVA with Tukey's post hoc comparison of means,
*P < 0.05.

TABLE 38
Liver and skeleton burden of ²⁴¹Am after seven
days following a single dose treatment.

| | | | | % of | | % of |
|---|---|---|---|---|---|---|
| | | | Liver | Reduction | Skeleton | Reduction |
| | | | Burden | over | Burden^{a} | over |
| Treatment | Route | N | (% of ID) | control | (% of ID) | control |
| Control | — | 3 | 16.5 ± 2.2 | — | 16.4 ± 4.1 | — |
| Ca-DTPA | i.v. | 4 | 10.2 ± 2.7* | 38.2 | 11.6 ± 2.2^{b} | 29.3 |
| 14 mg/kg | | | | | | |
| C2E5 | Transdermal | 3 | 8.4 ± 2.0* | 49.1 | 9.6 ± 0.9* | 41.1 |
| 1000 mg/kg | | | | | | |

One-way ANOVA with Tukey's post hoc comparison of means,
*P < 0.05.
^{a}Skeleton burden was calculated based on formula Skeleton burden = ²⁴¹Am burden of contra-lateral femur × 19 + ²⁴¹Am burden of ipsi-lateral femur.
^{b}One animal in IV Ca-DTPA treatment group was excluded from skeleton burden calculation (n = 3) due to extremely high ²⁴¹Am content (13.5% compared to 0.5 to 2.5% range for the rest of 9 tested animals) possibly as a result of deep IV injection which may have contaminated ipsi-lateral femur.

A method of preparing a nonaqueous gel of a multi-ester prodrug was developed for delivery of a radionuclide chelator after topical administration. In vitro metabolism and percutaneous permeation studies demonstrated that C2E5 can be released from the nonaqueous gel matrix and be de-esterified by endogenous skin esterases. An enhancement of total decorporation and reduction in liver and skeleton burden of ²⁴¹Am was observed after administration of the C2E5 transdermal gel to contaminated rats. The enhancement of ²⁴¹Am elimination for a period of at least 3 days indicated that the C2E5 nonaqueous gel formulation provided sustained delivery of DTPA and other C2E5 metabolites capable of chelating radionuclides. No skin abnormalities or signs of skin irritation were observed after administration of the C2E5 nonaqueous gel throughout the entire study period. The effectiveness of this treatment option, favorable sustained release profile of chelators, and ease of administration support this formulation as a candidate for inclusion in the Strategic National Stockpile for radiological and nuclear emergencies.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

1. A non-aqueous topical formulation comprising:
a compound of Formula (I): 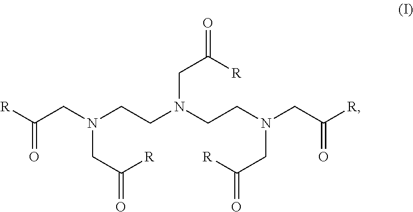
wherein:
R is —OR¹ or —NHR¹;
R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen.

2. The non-aqueous topical formulation of claim 1, wherein the compound of Formula (I) is present in an amount of about 10% to about 50% by weight of the formulation.

3. The non-aqueous topical formulation of claim 1, wherein R is —OR¹, optionally wherein R¹ is C₁-C₃₀ alkyl.

4. (canceled)

5. The non-aqueous topical formulation of claim 1, wherein the compound of Formula (I) has the following structure:

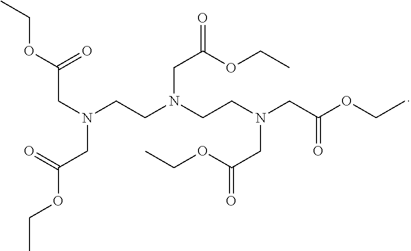 [SMILES: CCOC(=O)CN(CCN(CC(=O)OCC)CC(=O)OCC)CCN(CC(=O)OCC)CC(=O)OCC]

6. The non-aqueous topical formulation of claim 1, further comprising a hydrophobic polymer, optionally wherein the hydrophobic polymer is present in an amount of about 5% to about 40% by weight of the formulation.

7.-8. (canceled)

9. The non-aqueous topical formulation of claim 1, further comprising a fatty acid ester, optionally wherein the fatty acid ester is present in an amount of about 20% to about 60% by weight of the formulation.

10.-11. (canceled)

12. The non-aqueous topical formulation of claim 1, wherein the formulation has a water content of less than about 1% by weight of the formulation.

13. The non-aqueous topical formulation of claim 1, wherein the formulation comprises a gel.

14. The non-aqueous topical formulation of claim 1, wherein the formulation is prepared using a solvent evaporation method.

15. A topical formulation comprising:
a hydrophobic polymer;
a fatty acid ester; and
a compound of Formula (I): 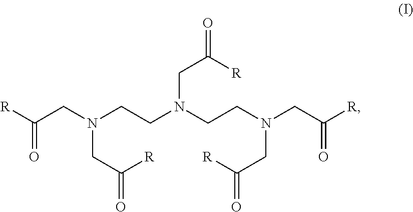
wherein:
R is —OR¹ or —NHR¹;
R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen.

16. The topical formulation of claim 15, wherein the compound of Formula (I) is present in an amount of about 10% to about 50% by weight of the formulation.

17. The topical formulation of claim 15, wherein R is —OR¹, optionally wherein R¹ is C₁-C₃₀ alkyl.

18. (canceled)

19. The topical formulation of claim 15, wherein the compound of Formula (I) has the following structure:

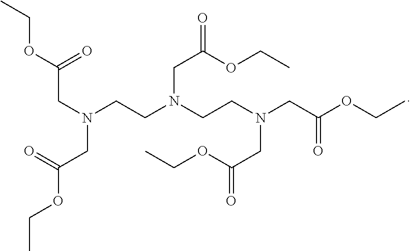 [SMILES: CCOC(=O)CN(CCN(CC(=O)OCC)CC(=O)OCC)CCN(CC(=O)OCC)CC(=O)OCC]

20. The topical formulation of claim 15, wherein the hydrophobic polymer is present in an amount of about 5% to about 40% by weight of the formulation.

21. (canceled)

22. The topical formulation of claim 15, wherein the fatty acid ester is present in an amount of about 20% to about 60% by weight of the formulation.

23. (canceled)

24. The topical formulation of claim 15, wherein the formulation is non-aqueous.

25. The topical formulation of claim 24, wherein the formulation has a water content of less than about 1% by weight of the formulation.

26. The topical formulation of claim 15, wherein the formulation comprises a gel.

27. The topical formulation of claim 15, wherein the formulation is prepared using a solvent evaporation method.

28. A method of treating a subject to remove a radioactive element from the subject comprising:
administering a therapeutically effective amount of a topical formulation to a subject, wherein the topical formulation comprises
a compound of Formula (I): 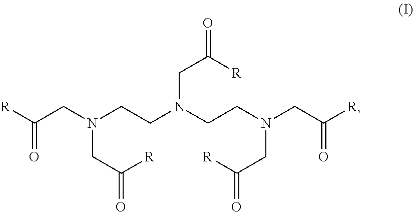
wherein:
R is —OR¹ or —NHR¹;
R¹ is each independently selected from the group consisting of H, C₁-C₃₀ alkyl, C₂-C₃₀ alkenyl, C₂-C₃₀ alkynyl, benzyl, cycloalkyl, aryl, arylalkyl, arylalkenyl, arylalkynyl, heterocyclic, and amino acid derivative and wherein when R is —OR¹ at least one R¹ is not hydrogen, and
wherein the topical formulation is non-aqueous.

29.-40. (canceled)
